(19) United States
(12) Patent Application Publication
(10) Pub. No.: US 2004/0106663 A1
(43) Pub. Date: Jun. 3, 2004

(54) Inhibitors of fungal invasion

(76) Inventors: John Jeffrey Talley, Somerville, MA (US); Angelika Fretzen, Somerville, MA (US); Craig Zimmerman, Topsfield, MA (US); Timothy Barden, Salem, MA (US); Jing Jing Yang, Boxborough, MA (US); Eduardo Martinez, Cambridge, MA (US); Robert Busby, Weymouth, MA (US); Etchell A. Cordero, North Reading, MA (US); Fariba Houman Cipriano, Belmont, MA (US); Christine M. Pierce, Tewksbury, MA (US); Eric F. Summers, Brookline, MA (US)

Correspondence Address: FISH & RICHARDSON PC, 225 FRANKLIN ST, BOSTON, MA, 02110, US

(21) Appl. No.: 10/657,753

(22) Filed: Sep. 8, 2003

Related U.S. Application Data
(60) Provisional application No. 60/408,561, filed on Sep. 6, 2002.
(60) Provisional application No. 60/443,693, filed on Jan. 30, 2003.

(51) Int. Cl. A61K 31/42; C07D 261/14

(52) U.S. Cl. 514/378; 548/246

(57) ABSTRACT

This invention relates to isoxazole-based inhibitors of fungal invasion.

### CROSS REFERENCE TO RELATED APPLICATIONS

This application claims the benefit of U.S. Provisional Application No. 60/408,561, filed on Sep. 6, 2002 and U.S. Provisional Application No. 60/443,693, filed on Jan. 30, 2003. The contents of both of these applications is incorporated herein by reference in its entirety.

### BACKGROUND

Fungal infections are a serious health concern, particularly for patients whose immune systems have been compromised by disease, chemotherapy, or immunosuppressive drugs. The frequency of Candida infections has increased in recent years and has been accompanied by a significant rise in morbidity and mortality. Candidiasis, which is most often caused by the pathogenic yeast Candida albicans, is the most frequent fungal infection associated with AIDS and other immunocompromised states. Many of these infections take place in the hospital setting. A majority of nosocomial septicemias caused by Candida species derive from biofilm formation on catheters and shunts.

A wide variety of plant-pathogenic fungi (e.g., blights, rusts, molds, smuts, and mildews) cause huge food crop loss and damage to ornamental plants. Plant diseases are caused by a myriad of invasive fungal pathogens falling into many genera, for example, soft rot (e.g., Rhizopus), leaf curl (e.g., Taphrina), powdery mildew (e.g., Sphaerotheca), leaf spots (e.g., Fulvia), blight (e.g., Alternaria), blast (e.g., Magnaporthe), black rot (e.g., Guignardia), scab (e.g., Venturia), wilts (e.g., Fusarium), rusts (e.g., Puccinia), smuts (e.g., Ustilago), and cankers (e.g., Rhizoctonia).

Recently, there has been great interest in identifying genes that may be implicated as important virulence factors in these infections. The virulence of Candida albicans has been shown to be dependent upon invasion of host tissues; mutations in any of several genes required for invasive growth substantially reduce virulence in a mouse model of systemic infection.

The SSK1 response regulator gene from C. albicans is essential for normal hyphal development and virulence. Cos1, a two-component histidine kinase, is required for normal hyphal growth of C. albicans, and may play a role in virulence properties of the organism. Deletion of the C. albicans gene encoding the mitogen-activated protein kinase HOG1 causes derepression of serum induced hyphal formation and a dramatic increase in the survival time of systemically infected mice. Disruption of the C. albicans mitogen activated protein kinase CEK1 adversely affects the growth of serum induced mycelial colonies and attenuates virulence in a mouse model for systemic candidiasis. These and other studies have suggested that hyphal growth may be an important virulence factor in C. albicans. Nonfilamentous C. albicans mutants are avirulent.

The exact mechanism by which hyphal growth acts as a virulence factor is also not known with certainty, but it is believed that there is a correlation between germ tube length and organ invasion in C. albicans clinical isolates. C. albicans may resist intracellular killing by macrophages through the formation of germ tubes.

A variety of antifungal compounds have been developed, some of which also affect hyphal growth. But there is a need for less toxic treatment regimens than those presently available. For example, over 5% of patients treated with fluconazole had adverse reactions, possibly related to the treatment, about half of which necessitated discontinuation of therapy. There is also a need for effective anti-Candida agents having fewer toxicological problems than amphotericin B, which by virtue of their lower toxicities can be administered to high risk patients either prophylactically or at the earliest signs of infection, without the need for a firm diagnosis.

### SUMMARY

The invention features compounds useful in the therapeutic or prophylactic treatment of fungal infection. Examples of fungi which cause fungal infections in humans include, without limitation, Absidia spp., Absidia corymbifera, Ajellomyces capsulatus, Ajellomyces dermatitidis, Allescheria boydii, Alternaria spp., Anthopsis deltoidea, Aphanomyces spp., Apophysomyces elequans, Armillaria spp., Arnium leoporinum, Arthroderma benhamiae, Arthroderma fulvum, Arthroderma gypseum, Arthroderma incurvatum, Arthroderma otae, Arthroderma vanbreuseghemii, Aspergillus spp., Aspergillus flavus, Aspergillus fumigatus, Aspergillus niger, Aureobasidium pullulans, Basisdiobolus ranarum, Bipolaris spp., Blastomyces dermatitidis, Botrytis spp., Candida spp., Candida albicans, Candida glabrata, Candida guilliermondii, Candida kefyr, Candida krusei, Candida parapsilosis, Candida pelliculosa, Candida tropicalis, Centrospora spp., Cephalosporium spp., Ceratocystis spp., Chaetoconidium spp., Chaetomium spp., Cladophialophora carrionii, Cladosporium spp., Coccidioides immitis, Colletotrichium spp., Conidiobolus spp., Cryptoporiopsis spp., Cylindrocladium spp., Cryptococcus spp., Cryptococcus neoformans, Cunninghamella spp., Cunninghamella bertholletiae, Curvularia spp., Dactylaria spp., Diplodia spp., Epidermophyton spp., Epidermophyton floccosum, Exserophilium spp., Exophiala spp., Exophiala dermatitidis, Filobasidiella neoformans, Fonsecaea spp., Fonsecaea pedrosoi, Fulvia spp., Fusarium spp., Fusarium solani, Geotrichum spp., Geotrichum candidum, Guignardia spp., Helminthosporium spp., Histoplasma spp., Histoplasma capsulatum, Hortaea werneckii, Issatschenkia orientalis, Lecythophora spp., Macrophomina spp., Madurella spp., Madurella grisae, Magnaporthe spp., Malassezia furfur, Malassezia globosa, Malassezia obtuse, Malassezia pachydermatis, Malassezia restricta, Malassezia slooffiae, Malassezia sympodialis, Microsporum spp., Microsporum canis, Microsporum fulvum, Microsporum gypseum, Monilinia spp., Mucor spp., Mucor circinelloides, Mycocentrospora acerina, Nectria spp., Nectria haematococca, Nocardia spp., Oospora spp., Ophiobolus spp., Paecilomyces spp., Paecilomyces variotii, Paracoccidioides brasiliensis, Penicillium spp., Penicillium marneffei, Phaeosclera dematioides, Phaeoannellomyces spp., Phialemonium obovatum, Phialophora spp., Phlyctaena spp., Phoma spp., Phomopsis spp., Phymatotrichum spp., Phytophthora spp., Pichia anomala, Pichia guilliermondii, Pythium spp., Piedraia hortai, Pneumocystis carinii, Pseudallescheria boydii, Puccinia spp., Pythium insidiosum, Rhinocladiella aquaspersa, Rhizomucor pusillus, Rhizoctonia spp., Rhizopus spp., Rhizopus oryzae, Rhodotorula rubra, Saccharomyces spp., Saccharomyces cerevisiae, Saksenaea vasiformis, Sarcinomyces phaeomuriformis, Scedosporium apiospermum, Scerotium spp., Schizophyllum commune, Sclerotinia spp., Sphaerotheca spp., Sporothrix schenckii, Syncephalastrum racemosum, Taeniolella boppii, Taphrina spp., Thielaviopsis spp., Torulopsis spp., Trichophyton spp., Trichophyton mentagrophytes, Trichophyton rubrum, Trichophyton verrucosum, Trichophyton violaceum, Trichosporon spp., Trichosporon asahii, Trichosporon cutaneum, Trichosporon inkin, Trichosporon mucoides, Ulocladium chartarum, Ustilago spp., Venturia spp., Verticillium spp., Wangiella dermatitidis, Whetxelinia spp., and Xylohypha spp.

Examples of fungi that cause infections in animals include, without limitation, Alternaria spp., Aspergillus spp. Candida spp., Cladosporium spp., Geotrichum spp., Microsporum canis, Microsporum eguinum, Microsporum gallinae, Microsporum nanum, Paecilomyces spp., Penicillium spp., Trichophyton mentagrophytes, and Trichophyton verucosum.

Certain compounds described herein inhibit fungal invasion. The compounds may also be useful for treating, either therapeutically or prophylactically, fungal infections that are not invasive. Preferred compounds are substantially non-toxic to a mammal at dosages that are effective for inhibiting fungal invasion in vivo.

In one aspect, this invention relates to a compound having a formula (I):

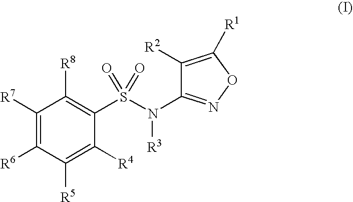

or a pharmaceutically acceptable salt thereof, wherein,

each of R¹ and R² is, independently, H, substituted or unsubstituted C₁₋₆ alkyl, or substituted or unsubstituted C₁₋₆ alkoxy, wherein the substituents are selected from the group consisting of hydroxy and halo;

R³ is H, formyl, acetyl, or substituted or unsubstituted C₁₋₃ alkyl, wherein the substituents are selected from the group consisting of hydroxy and halo;

each of R⁴-R⁸ is, independently, H, halo, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyl)oxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, —NH—C(O)—NH-(substituted or unsubstituted heteroaryl), or —NR¹⁹R²⁰, wherein each of R¹⁹ and R²⁰ is, independently, H, C₁₋₁₂ alkyl, or C₂₋₁₂ alkenyl, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxy(C₁₋₆)alkyloxy, (C₁₋₆) acylamino, (C₁₋₆) acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy; provided that at least one of R⁴-R⁸ is not H; further provided that when R¹ is alkyl, then R⁶ is not butyl; further provided that when R¹ is methyl and R² is H, then R⁶ is not —C≡CH, —NHCH₃, CF₃, or —CH₂CH₃.

Embodiments can include one or more of the following.

R¹ can be C₁-C₄ alkyl (e.g., CH₃).

R⁴, R⁵, R⁷, and R⁸ can be H.

R³ can be H.

R⁶ can be C₁-C₁₀ alkyl, e.g., cyclopentyl, norbornyl.

R⁶ can be C₁-C₁₀ alkoxy.

R⁶ can be substituted or unsubstituted C₆-C₁₀ aryl.

R⁶ is substituted or unsubstituted C₂-C₁₂ alkenyl.

Each of R⁴—R⁸ can be, independently, H, halo, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, substituted or unsubstituted phenyl, substituted or unsubstituted heteroaryl, or —NH—(C₁₋₆ alkyl), wherein the substituents are selected from the group consisting of hydroxy, halo, and C₁₋₄ alkyl. In certain embodiments, R⁴, R⁵, R⁷, and R⁸ can be H. In other embodiments, when R¹ is alkyl, then R⁶ cannot be alkyl; or when R¹ is methyl and R² is H, then R⁶ cannot be alkyl; or when R¹ is methyl and R² is H, then R⁶ cannot be alkynyl; or when R¹ is methyl and R² is H, then R⁶ cannot be —NH(C₁-C₆ alkyl). In still other embodiments, R³ can be H.

In another aspect, this invention features a compound having a formula (II):

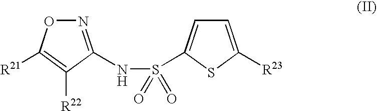

or a pharmaceutically acceptable salt thereof, wherein

each of R²¹ and R²² is, independently, substituted or unsubstituted C₁₋₆ alkyl, or substituted or unsubstituted C₁₋₆ alkoxy, wherein the substituents are selected from the group consisting of hydroxy and halo;

R²³ is substituted or unsubstituted C₁₋₆ alkyl, substituted or unsubstituted C₆₋₁₂ aryl, substituted or unsubstituted C₃₋₁₂ heteroaryl, wherein the substituents are selected from the group consisting of halo, C₁₋₆ alkyl, and C₁₋₆ trihaloalkyl. R²¹ can be C₁-C₄ alkyl, e.g., CH₃.

In a further aspect, this invention features a pharmaceutical composition that contains an amount (e.g., an effective amount) of at least one of the compounds described above e.g. a compound having the formula (I) or (II) and a pharmaceutically acceptable carrier. In certain embodiments, the composition can contain a second antimicrobial agent.

In one aspect, this invention features a method of treating (therapeutically or prophylactically) a fungal infection in a subject (including a subject identified as in need of such treatment), the method includes administering an effective amount of a compound, e.g. a compound having the formula (I) or (II) or a pharmaceutical composition described above to the subject. In certain embodiments, the method can include administering a compound or a pharmaceutical composition described above to the subject in combination with a second antimicrobial agent.

In another aspect, this invention relates to the use of a compound having the formula (I) or (II) in medicine.

In another aspect, this invention relates to the use of a compound having the formula (I) or (II) in the manufacture of a medicament for the therapeutic or prophylactic treatment of a fungal infection.

Also within the invention are the compounds shown in Tables 1 and 2. The invention also features pharmaceutical compositions containing the compounds described above and those in Tables 1 and 2. In some cases, hydrogen atoms on the sulfonamide nitrogens have been omitted for clarity.

TABLE 1

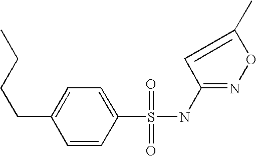

4-butyl-N-(5-methylisoxazol-3-yl)benzenesulfonamide

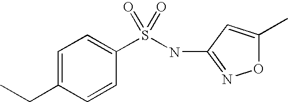

4-ethyl-N-(5-methylisoxazol-3-yl)benzenesulfonamide

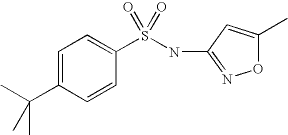

4-tert-butyl-N-(5-methylisoxazol-3-yl)benzenesulfonamide

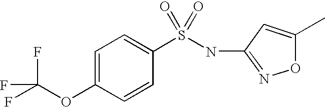

N-(5-methylisoxazol-3-yl)-4-(trifluoromethoxy)benzenesulfonamide

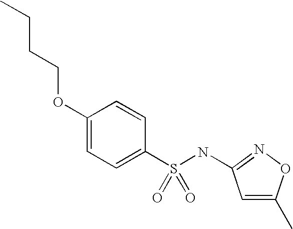

4-butoxy-N-(5-methylisoxazol-3-yl)benzenesulfonamide

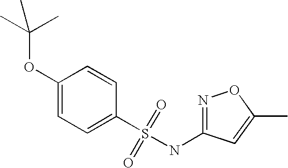

4-tert-butoxy-N-(5-methylisoxazol-3-yl)benzenesulfonamide

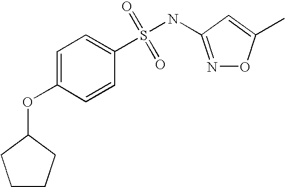

4-(cyclopentyloxy)-N-(5-methylisoxazol-3-yl)benzenesulfonamide

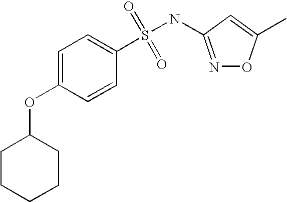

4-(cyclohexyloxy)-N-(5-methylisoxazol-3-yl)benzenesulfonamide

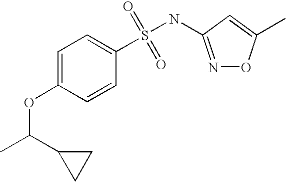

4-(1-cyclopropylethoxy)-N-(5-methylisoxazol-3-yl)benzenesulfonamide

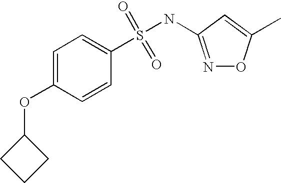

4-(cyclobutyloxy)-N-(5-methylisoxazol-3-yl)benzenesulfonamide

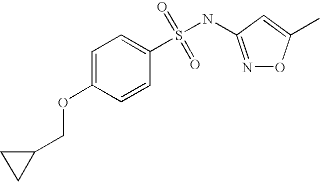

4-(cyclopropylmethoxy)-N-(5-methylisoxazol-3-yl)benzenesulfonamide

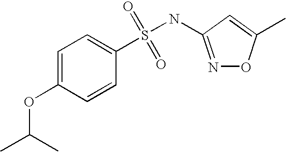

4-isopropoxy-N-(5-methylisoxazol-3-yl)benzenesulfonamide

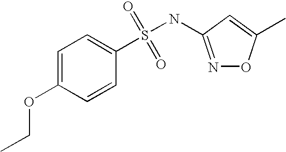

4-ethoxy-N-(5-methylisoxazol-3-yl)benzenesulfonamide

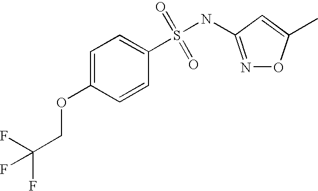

N-(5-methylisoxazol-3-yl)-4-(2,2,2-trifluoroethoxy)benezenesulfonamide

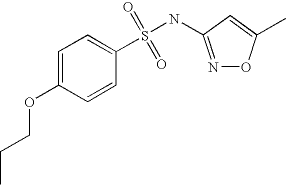

N-(5-methylisoxazol-3-yl)-4-propoxybenzenesulfonamide

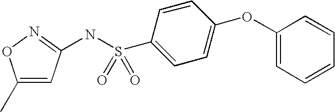

N-(5-methylisoxazol-3-yl)-4-phenoxybenzenesulfonamide

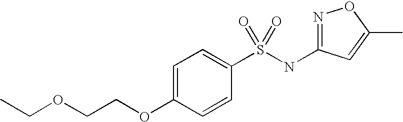

4-(2-ethoxyethoxy)-N-(5-methylisoxazol-3-yl)benzenesulfonamide

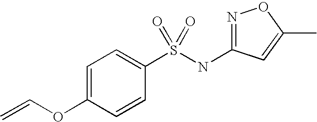

N-(5-methylisoxazol-3-yl)-4-(vinyloxy)benzenesulfonamide

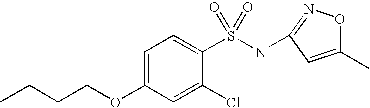

4-butoxy-2-chloro-N-(5-methylisoxazol-3-yl)benzenesulfonamide

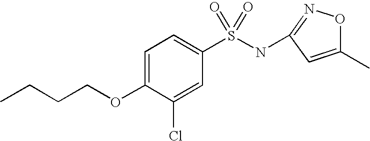

4-butoxy-3-chloro-N-(5-methylisoxazol-3-yl)benzenesulfonamide

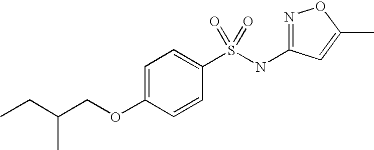

4-(2-methylbutoxy)-N-(5-methylisoxazol-3-yl)benzenesulfonamide

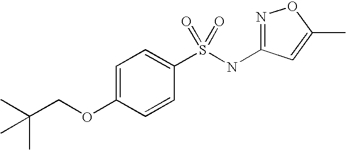

N-(5-methylisoxazol-3-yl)-4-(neopentyloxy)benzenesulfonamide

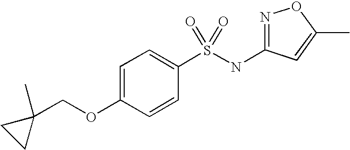

4-[(1-methylcyclopropyl)methoxy]-N-(5-methylisoxazol-3-
yl)benzenesulfonamide

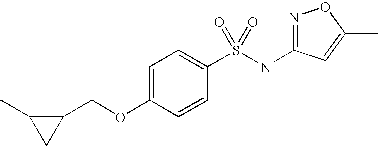

4-[(2-methylcyclopropyl)methoxy]-N-(5-methylisoxazol-3-
yl)benzenesulfonamide

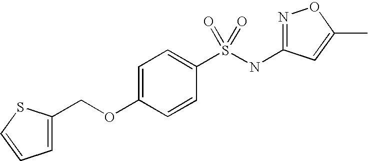

N-(5-methylisoxazol-3-yl)-4-(thien-2-ylmethoxy)benzenesulfonamide

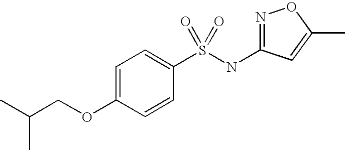

4-isobutoxy-N-(5-methylisoxazol-3-yl)benzenesulfonamide

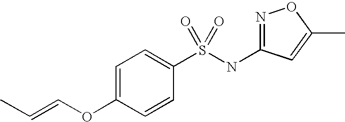

N-(5-methylisoxazol-3-yl)-4-[(1E)-prop-1-enyloxy]benzenesulfonamide

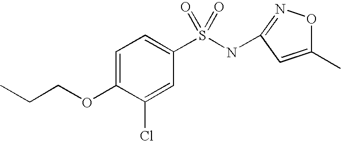

3-chloro-N-(5-methylisoxazol-3-yl)-4-propoxybenzenesulfonamide

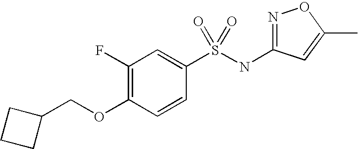

4-(cyclobutylmethoxy)-3-fluoro-N-(5-methylisoxazol-3-
yl)benzenesulfonamide

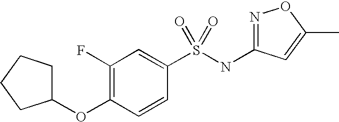

4-(cyclopentyloxy)-3-fluoro-N-(5-methylisoxazol-3-
yl)benzenesulfonamide

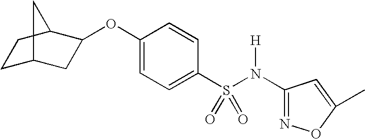

4-(bicyclo[2.2.1]hept-2-yloxy)-N-(5-methylisoxazol-3-
yl)benzenesulfonamide

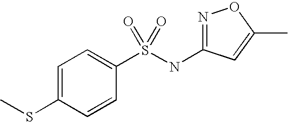

N-(5-methylisoxazol-3-yl)-4-(methylthio)benzenesulfonamide

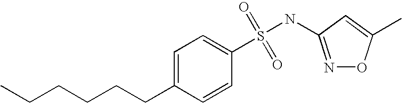

4-hexyl-N-(5-methylisoxazol-3-yl)benzenesulfonamide

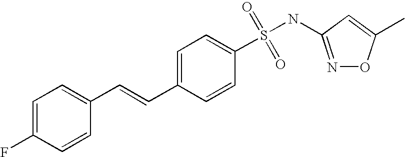

4-[(E)-2-(4-fluorophenyl)vinyl]-N-(5-methylisoxazol-3-
yl)benzenesulfonamide

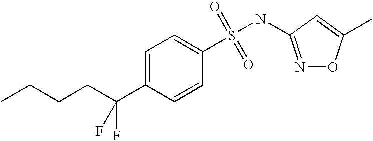

4-(1,1-difluoropentyl)-N-(5-methylisoxazol-3-yl)benzenesulfonamide

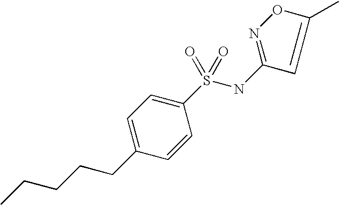

N-(5-methylisoxazol-3-yl)-4-pentylbenzenesulfonamide

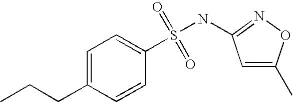

N-(5-methylisoxazol-3-yl)-4-propylbenzenesulfonamide

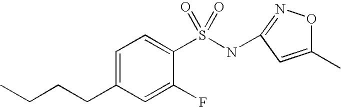

4-butyl-2-fluoro-N-(5-methylisoxazol-3-yl)benzenesulfonamide

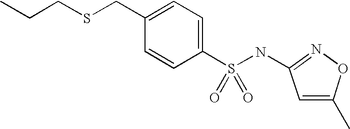

N-(5-methylisoxazol-3-yl)-4-[(propylthio)methyl]benzenesulfonamide

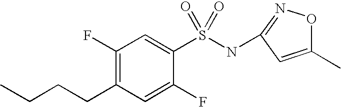

4-butyl-2,5-difluoro-N-(5-methylisoxazol-3-yl)benzenesulfonamide

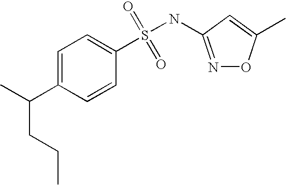

4-(1-methylbutyl)-N-(5-methylisoxazol-3-yl)benzenesulfonamide

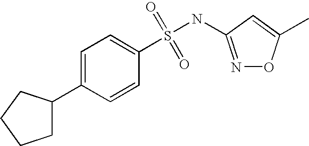

4-cyclopentyl-N-(5-methylisoxazol-3-yl)benzenesulfonamide

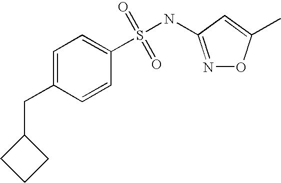

4-(cyclobutylmethyl)-N-(5-methylisoxazol-3-yl)benzenesulfonamide

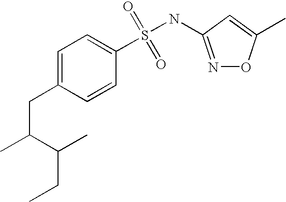

4-(2,3-dimethylpentyl)-N-(5-methylisoxazol-3-yl)benzenesulfonamide

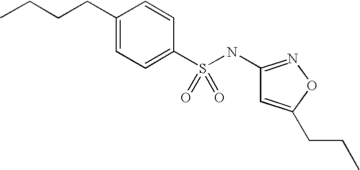

4-butyl-N-(5-propylisoxazol-3-yl)benzenesulfonamide

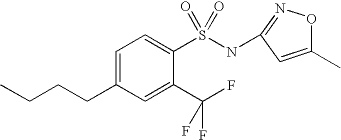

4-butyl-N-(5-methylisoxazol-3-yl)-2-(trifluoromethyl)benzenesulfonamide

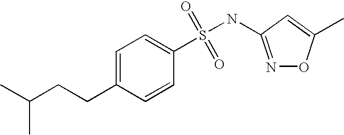

4-(3-methylbutyl)-N-(5-methylisoxazol-3-yl)benzenesulfonamide

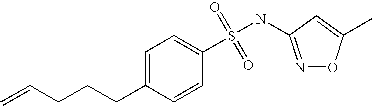

N-(5-methylisoxazol-3-yl)-4-pent-4-enylbenzenesulfonamide

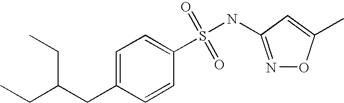

4-(2-ethylbutyl)-N-(5-methylisoxazol-3-yl)benzenesulfonamide

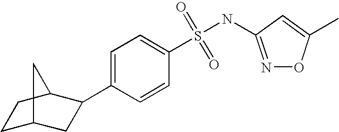

4-bicyclo[2.2.1]hept-2-yl-N-(5-methylisoxazol-3-yl)benzenesulfonamide

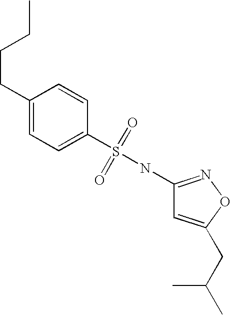

4-butyl-N-(5-isobutylisoxazol-3-yl)benzenesulfonamide

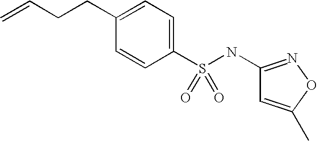

4-but-3-enyl-N-(5-methylisoxazol-3-yl)benzenesulfonamide

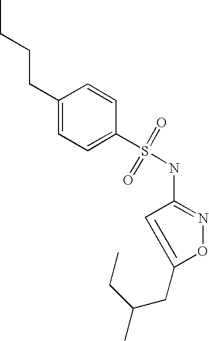

4-butyl-N-[5-(2-methylbutyl)isoxazol-3-yl]benzenesulfonamide

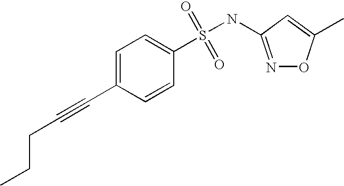

N-(5-methylisoxazol-3-yl)-4-pent-1-ynylbenzenesulfonamide

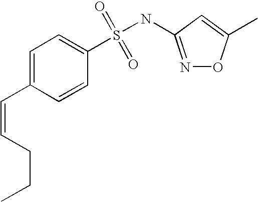

N-(5-methylisoxazol-3-yl)-4-[(1Z)-pent-1-enyl]benzenesulfonamide

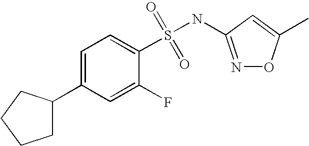

4-cyclopentyl-2-fluoro-N-(5-methylisoxazol-3-yl)benzenesulfonamide

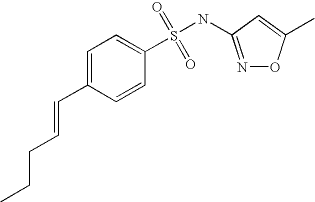

N-(5-methylisoxazol-3-yl)-4-[(1E)-pent-1-enyl]benzenesulfonamide

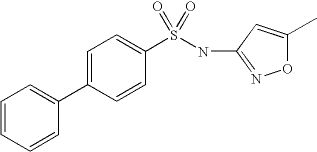

N-(5-methylisoxazol-3-yl)-1,1′-biphenyl-4-sulfonamide

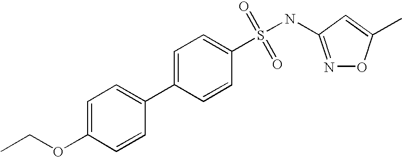

4′-ethoxy-N-(5-methylisoxazol-3-yl)-1,1′-biphenyl-4-sulfonamide

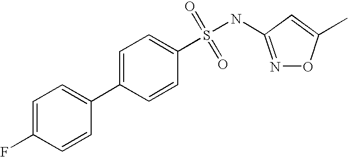

4′-fluoro-N-(5-methylisoxazol-3-yl)-1,1′-biphenyl-4-sulfonamide

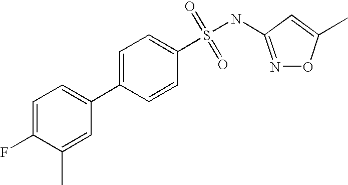

4′-fluoro-3′-methyl-N-(5-methylisoxazol-3-yl)-1,1′-biphenyl-4-sulfonamide

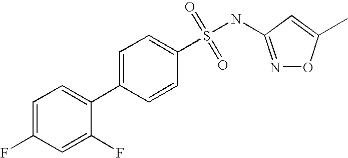

2′,4′-difluoro-N-(5-methylisoxazol-3-yl)-1,1′-biphenyl-4-sulfonamide

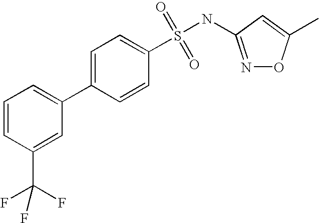

N-(5-methylisoxazol-3-yl)-3′-(trifluoromethyl)-1,1′-biphenyl-4-
sulfonamide

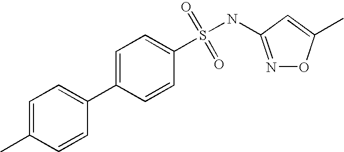

4′-methyl-N-(5-methylisoxazol-3-yl)-1,1′-biphenyl-4-sulfonamide

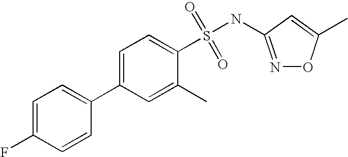

4′-fluoro-3-methyl-N-(5-methylisoxazol-3-yl)-1,1′-biphenyl-4-
sulfonamide

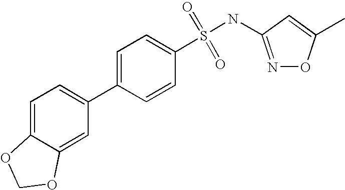

4-(1,3-benzodioxol-5-yl)-N-(5-methylisoxazol-3-yl)benzenesulfonamide

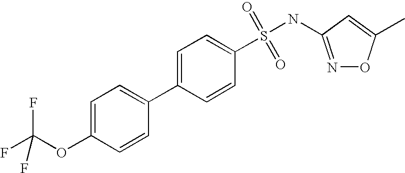

N-(5-methylisoxazol-3-yl)-4′-(trifluoromethoxy)-1,1′-biphenyl-4-
sulfonamide

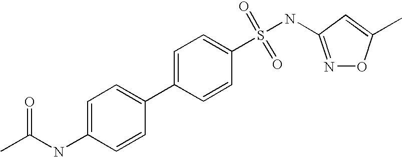

N-(4′-{[(5-methylisoxazol-3-yl)amino]sulfonyl}-1,1′-biphenyl-4-
yl)acetamide

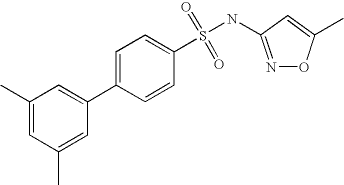

3′,5′-dimethyl-N-(5-methylisoxazol-3-yl)-1,1′-biphenyl-4-sulfonamide

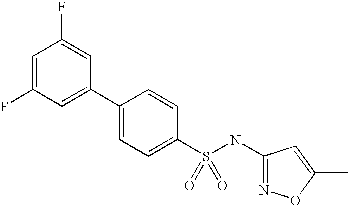

3′,5′-difluoro-N-(5-methylisoxazol-3-yl)-1,1′-biphenyl-4-sulfonamide

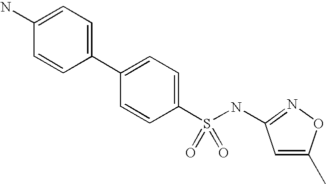

4′-amino-N-(5-methylisoxazol-3-yl)-1,1′-biphenyl-4-sulfonamide

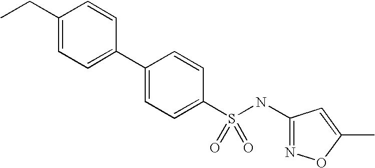

4′-ethyl-N-(5-methylisoxazol-3-yl)-1,1′-biphenyl-4-sulfonamide

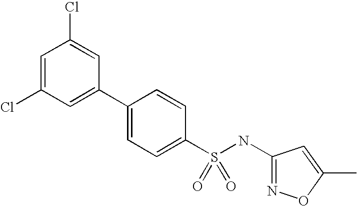

3′,5′-dichloro-N-(5-methylisoxazol-3-yl)-1,1′-biphenyl-4-sulfonamide

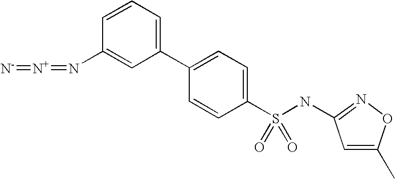

3′-azido-N-(5-methylisoxazol-3-yl)-1,1′-biphenyl-4-sulfonamide

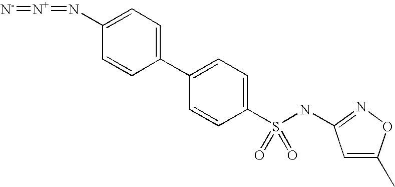

4′-azido-N-(5-methylisoxazol-3-yl)-1,1′-biphenyl-4-sulfonamide

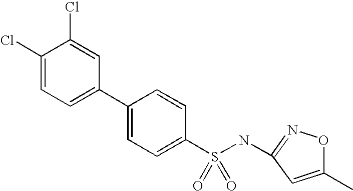

3′,4′-dichloro-N-(5-methylisoxazol-3-yl)-1,1′-biphenyl-4-sulfonamide

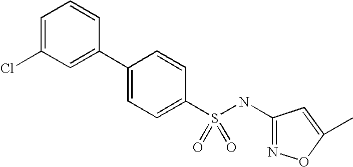

3′-chloro-N-(5-methylisoxazol-3-yl)-1,1′-biphenyl-4-sulfonamide

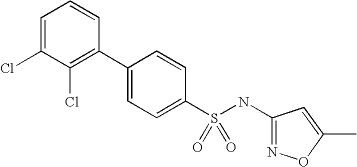

2′,3′-dichloro-N-(5-methylisoxazol-3-yl)-1,1′-biphenyl-4-sulfonamide

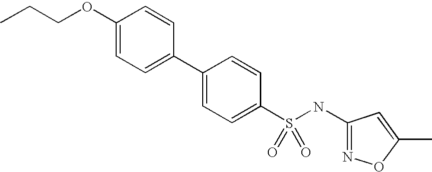

N-(5-methylisoxazol-3-yl)-4′-propoxy-1,1′-biphenyl-4-sulfonamide

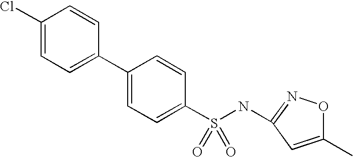

4′-chloro-N-(5-methylisoxazol-3-yl)-1,1′-biphenyl-4-sulfonamide

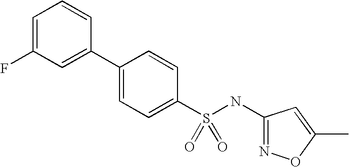

3′-fluoro-N-(5-methylisoxazol-3-yl)-1,1′-biphenyl-4-sulfonamide

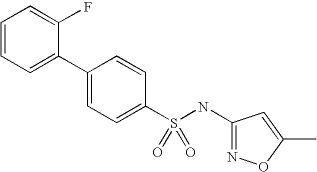

2′-fluoro-N-(5-methylisoxazol-3-yl)-1,1′-biphenyl-4-sulfonamide

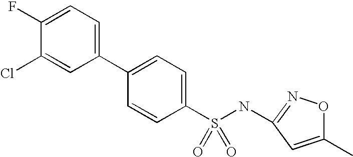

3′-chloro-4′-fluoro-N-(5-methylisoxazol-3-yl)-1,1′-biphenyl-4-sulfonamide

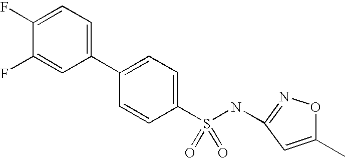

3′,4′-difluoro-N-(5-methylisoxazol-3-yl)-1,1′-biphenyl-4-sulfonamide

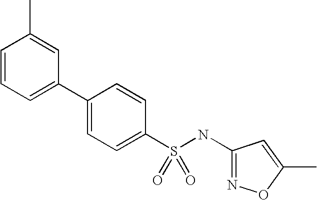

3′-methyl-N-(5-methylisoxazol-3-yl)-1,1′-biphenyl-4-sulfonamide

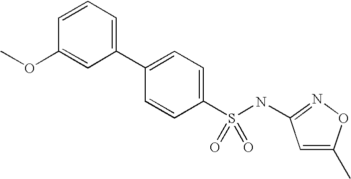

3′-methoxy-N-(5-methylisoxazol-3-yl)-1,1′-biphenyl-4-sulfonamide

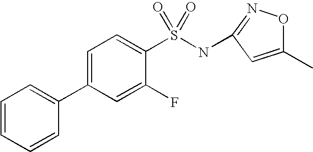

3-fluoro-N-(5-methylisoxazol-3-yl)-1,1′-biphenyl-4-sulfonamide

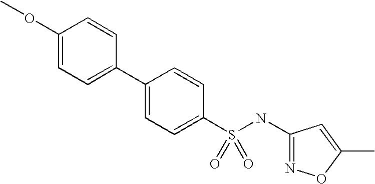

4′-methoxy-N-(5-methylisoxazol-3-yl)-1,1′-biphenyl-4-sulfonamide

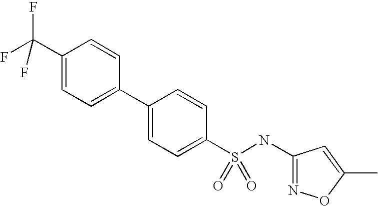

N-(5-methylisoxazol-3-yl)-4′-(trifluoromethyl)-1,1′-biphenyl-4-
sulfonamide

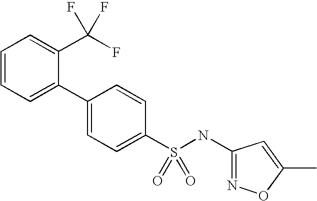

N-(5-methylisoxazol-3-yl)-2′-(trifluoromethyl)-1,1′-biphenyl-4-
sulfonamide

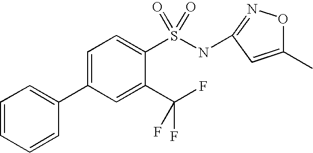

N-(5-methylisoxazol-3-yl)-3-(trifluoromethyl)-1,1′-biphenyl-4-sulfonamide

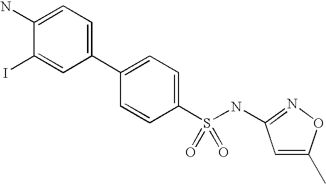

4′-amino-3′-iodo-N-(5-methylisoxazol-3-yl)-1,1′-biphenyl-4-sulfonamide

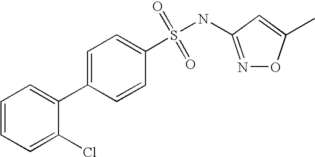

2′-chloro-N-(5-methylisoxazol-3-yl)-1,1′-biphenyl-4-sulfonamide

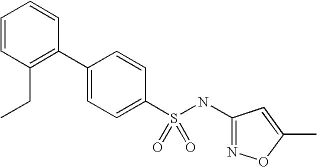

2′-ethyl-N-(5-methylisoxazol-3-yl)-1,1′-biphenyl-4-sulfonamide

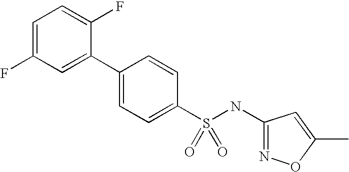

2′,5′-difluoro-N-(5-methylisoxazol-3-yl)-1,1′-biphenyl-4-sulfonamide

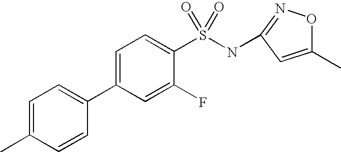

3-fluoro-4′-methyl-N-(5-methylisoxazol-3-yl)-1,1′-biphenyl-4-sulfonamide

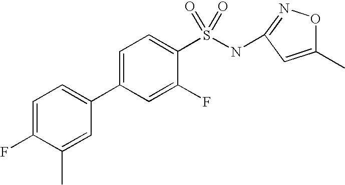

3,4′-difluoro-3′-methyl-N-(5-methylisoxazol-3-yl)-1,1′-biphenyl-4-
sulfonamide

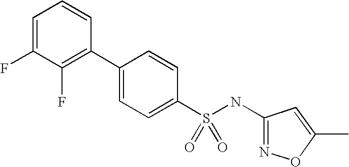

2′,3′-difluoro-N-(5-methylisoxazol-3-yl)-1,1′-biphenyl-4-sulfonamide

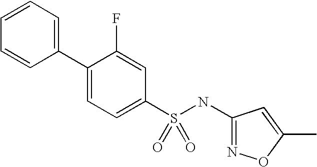

2-fluoro-N-(5-methylisoxazol-3-yl)-1,1′-biphenyl-4-sulfonamide

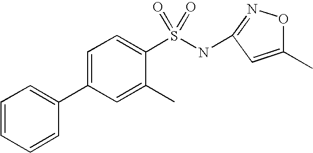

3-methyl-N-(5-methylisoxazol-3-yl)-1,1′-biphenyl-4-sulfonamide

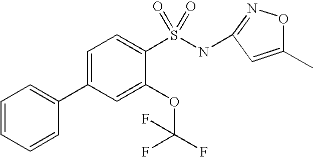

N-(5-methylisoxazol-3-yl)-3-(trifluoromethoxy)-1,1′-biphenyl-4-
sulfonamide

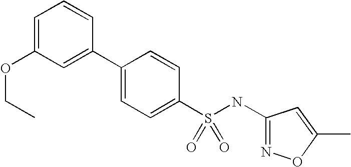

3′-ethoxy-N-(5-methylisoxazol-3-yl)-1,1′-biphenyl-4-sulfonamide

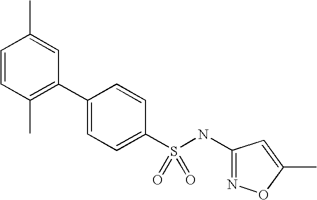

2′,5′-dimethyl-N-(5-methylisoxazol-3-yl)-1,1′-biphenyl-4-sulfonamide

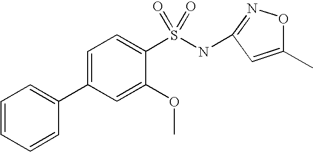

3-methoxy-N-(5-methylisoxazol-3-yl)-1,1′-biphenyl-4-sulfonamide

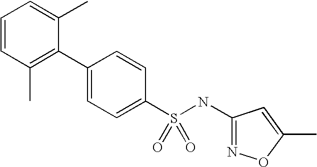

2′,6′-dimethyl-N-(5-methylisoxazol-3-yl)-1,1′-biphenyl-4-sulfonamide

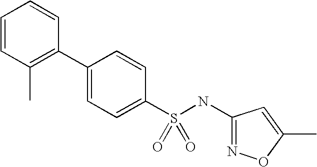

2′-methyl-N-(5-methylisoxazol-3-yl)-1,1′-biphenyl-4-sulfonamide

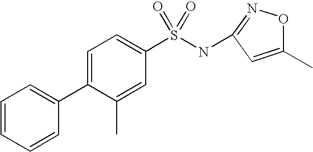

2-methyl-N-(5-methylisoxazol-3-yl)-1,1′-biphenyl-4-sulfonamide

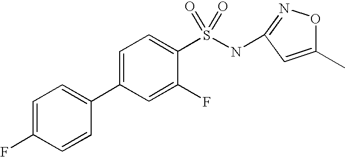

3,4′-difluoro-N-(5-methylisoxazol-3-yl)-1,1′-biphenyl-4-sulfonamide

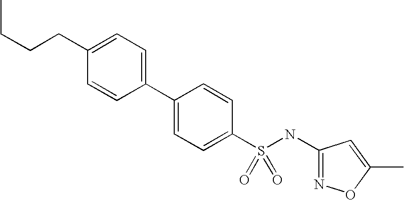

4′-butyl-N-(5-methylisoxazol-3-yl)-1,1′-biphenyl-4-sulfonamide

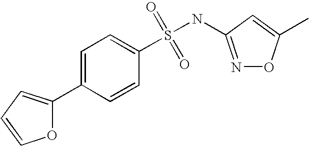

4-(2-furyl)-N-(5-methylisoxazol-3-yl)benzenesulfonamide

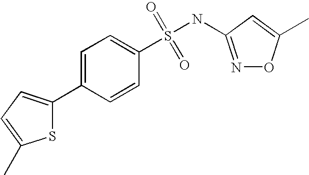

N-(5-methylisoxazol-3-yl)-4-(5-methylthien-2-yl)benzenesulfonamide

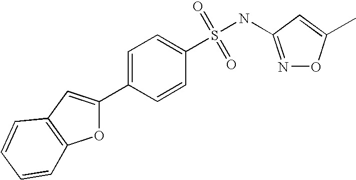

4-(1-benzofuran-2-yl)-N-(5-methylisoxazol-3-yl)benzenesulfonamide

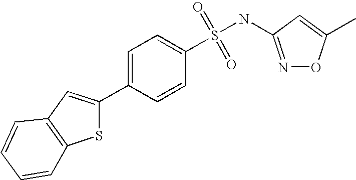

4-(1-benzothien-2-yl)-N-(5-methylisoxazol-3-yl)benzenesulfonamide

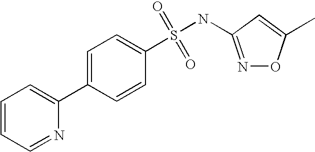

N-(5-methylisoxazol-3-yl)-4-pyridin-2-ylbenzenesulfonamide

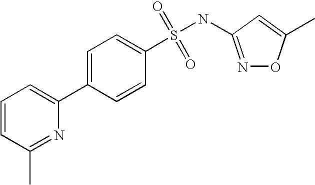

N-(5-methylisoxazol-3-yl)-4-(6-methylpyridin-2-yl)benzenesulfonamide

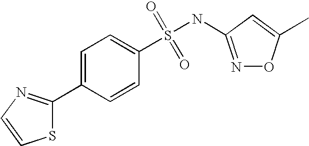

N-(5-methylisoxazol-3-yl)-4-(1,3-thiazol-2-yl)benzenesulfonamide

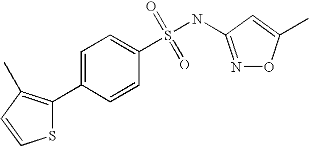

N-(5-methylisoxazol-3-yl)-4-(3-methylthien-2-yl)benzenesulfonamide

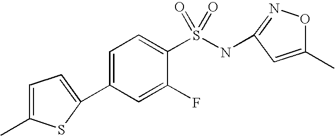

2-fluoro-N-(5-methylisoxazol-3-yl)-4-(5-methylthien-2-yl)benzene-
sulfonamide

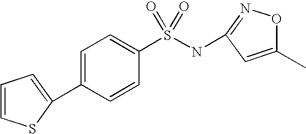

N-(5-methylisoxazol-3-yl)-4-thien-2-ylbenzenesulfonamide

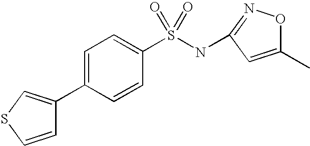

N-(5-methylisoxazol-3-yl)-4-thien-3-ylbenzenesulfonamide

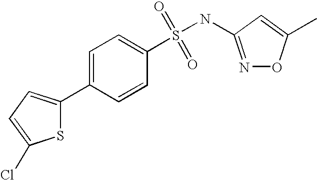

4-(5-chlorothien-2-yl)-N-(5-methylisoxazol-3-yl)benzenesulfonamide

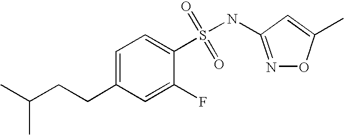

2-fluoro-4-(3-methylbutyl)-N-(5-methylisoxazol-3-yl)benzenesulfonamide

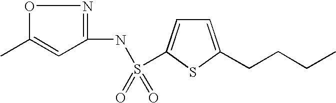

5-butyl-N-(5-methylisoxazol-3-yl)thiophene-2-sulfonamide

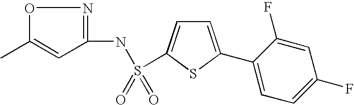

5-(2,4-difluorophenyl)-N-(5-methylisoxazol-3-yl)thiophene-2-sulfonamide

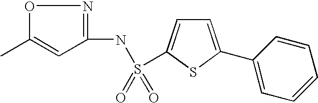

N-(5-methylisoxazol-3-yl)-5-phenylthiophene-2-sulfonamide

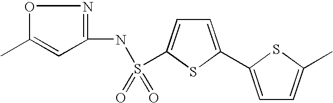

5′-methyl-N-(5-methylisoxazol-3-yl)-2,2′-bithiophene-5-sulfonamide

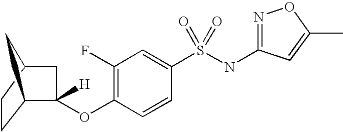

4-[(1S,2S)-bicyclo[2.2.1]hept-2-yloxy]-3-fluoro-N-(5-methylisoxazol-3-
yl)benzenesulfonamide

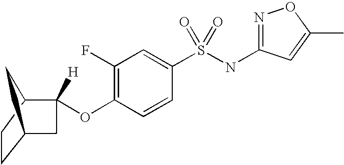

4-[(2R,4S)-bicyclo[2.2.1]hept-2-yloxy]-3-fluoro-N-(5-methylisoxazol-3-
yl)benzenesulfonamide

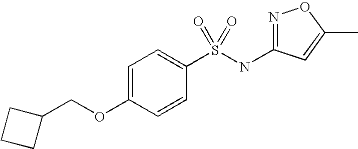

4-(cyclobutylmethoxy)-N-(5-methylisoxazol-3-yl)benzenesulfonamide

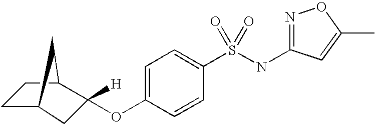

4-[(2R,4S)-bicyclo[2.2.1]hept-2-yloxy]-N-(5-methylisoxazol-3-
yl)benzenesulfonamide

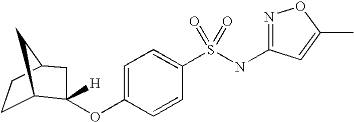

rac-4-[(1S,2S)-bicyclo[2.2.1]hept-2-yloxy]-N-(5-methylisoxazol-3-
yl)benzenesulfonamide

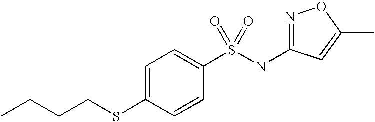

4-(butylthio)-N-(5-methylisoxazol-3-yl)benzenesulfonamide

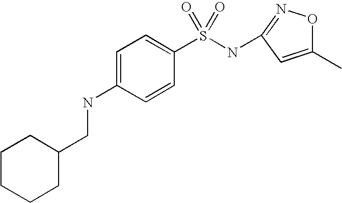

4-[(cyclohexylmethyl)amino]-N-(5-methylisoxazol-3-yl)benzene-
sulfonamide

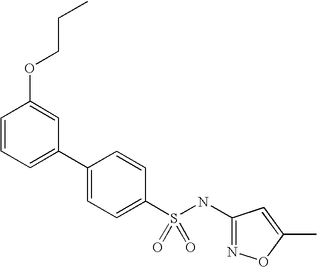

N-(5-methylisoxazol-3-yl)-3′-propoxy-1,1′-biphenyl-4-sulfonamide

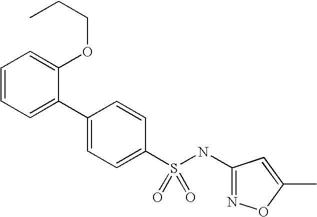

N-(5-methylisoxazol-3-yl)-2′-propoxy-1,1′-biphenyl-4-sulfonamide

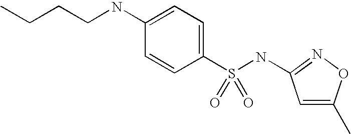

4-(butylamino)-N-(5-methylisoxazol-3-yl)benzenesulfonamide

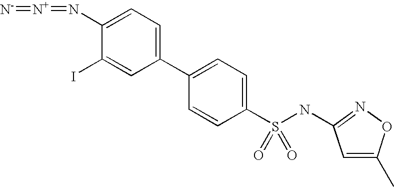

4′-azido-3′-iodo-N-(5-methylisoxazol-3-yl)-1,1′-biphenyl-4-sulfonamide

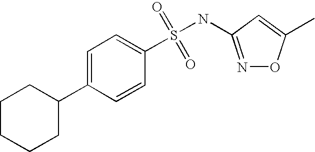

4-cyclohexyl-N-(5-methylisoxazol-3-yl)benzenesulfonamide

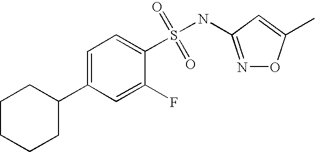

4-cyclohexyl-2-fluoro-N-(5-methylisoxazol-3-yl)benzenesulfonamide

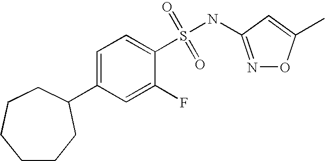

4-cycloheptyl-2-fluoro-N-(5-methylisoxazol-3-yl)benzenesulfonamide

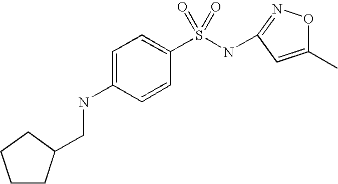

4-[(cyclopentylmethyl)amino]-N-(5-methylisoxazol-3-yl)benzene-
sulfonamide

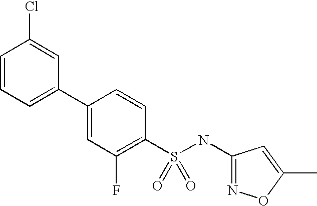

3′-chloro-3-fluoro-N-(5-methylisoxazol-3-yl)-1,1′-biphenyl-4-sulfonamide

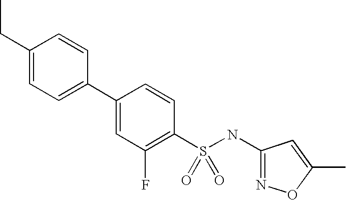

4′-ethyl-3-fluoro-N-(5-methylisoxazol-3-yl)-1,1′-biphenyl-4-sulfonamide

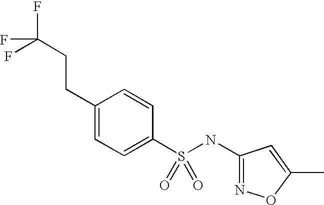

N-(5-methylisoxazol-3-yl)-4-(3,3,3-trifluoropropyl)benzenesulfonamide

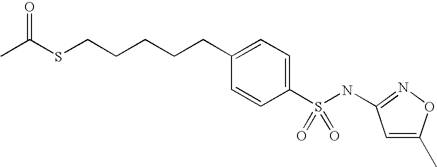

S-[5-(4-{[(5-methylisoxazol-3-yl)amino]sulfonyl}phenyl)pentyl]ethane-
thioate

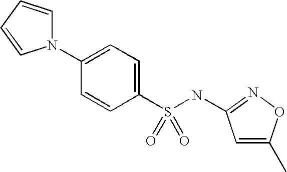

N-(5-methylisoxazol-3-yl)-4-(1H-pyrrol-1-yl)benzenesulfonamide

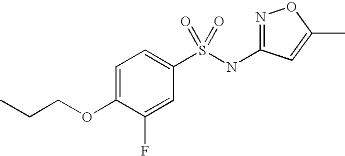

3-fluoro-N-(5-methylisoxazol-3-yl)-4-propoxybenzenesulfonamide

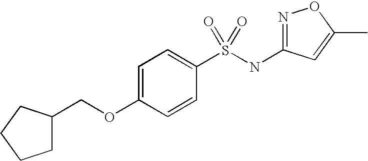

4-(cyclopentylmethoxy)-N-(5-methylisoxazol-3-yl)benzenesulfonamide

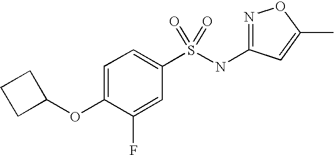

4-(cyclobutyloxy)-3-fluoro-N-(5-methylisoxazol-3-yl)benzenesulfonamide

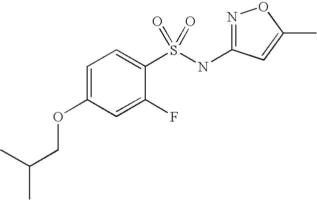

2-fluoro-4-isobutoxy-N-(5-methylisoxazol-3-yl)benzenesulfonamide

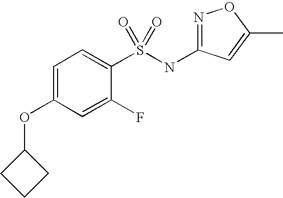

4-(cyclobutyloxy)-2-fluoro-N-(5-methylisoxazol-3-yl)benzenesulfonamide

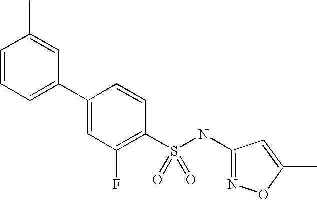

3-fluoro-3′-methyl-N-(5-methylisoxazol-3-yl)-1,1′-biphenyl-4-sulfonamide

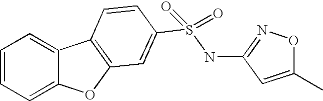

N-(5-methylisoxazol-3-yl)dibenzo[b,d]furan-3-sulfonamide

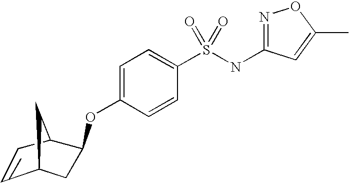

rac-4-[(2S,4S)-bicyclo[2.2.1]hept-5-en-2-yloxy]-N-(5-methylisoxazol-3-
yl)benzenesulfonamide

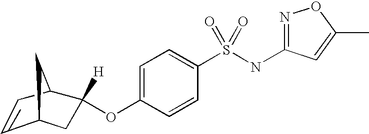

rac-4-[(2R,4S)-bicyclo[2.2.1]hept-5-en-2-yloxy]-N-(5-methylisoxazol-3-
yl)benzenesulfonamide

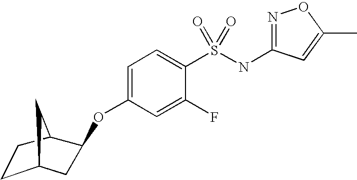

4-[(2S,4S)-bicyclo[2.2.1]hept-2-yloxy]-2-fluoro-N-(5-methylisoxazol-3-
yl)benzenesulfonamide

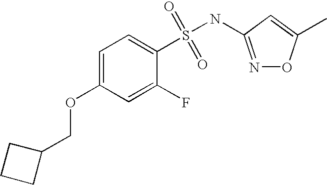

4-(cyclobutylmethoxy)-2-fluoro-N-(5-methylisoxazol-3-yl)benzene-
sulfonamide

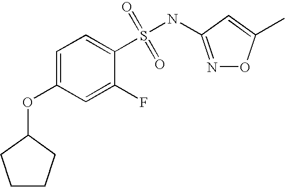

4-(cyclopentyloxy)-2-fluoro-N-(5-methylisoxazol-3-yl)benzene-
sulfonamide

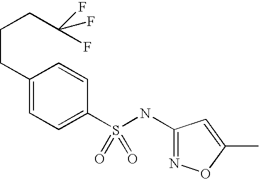

N-(5-methylisoxazol-3-yl)-4-(4,4,4-trifluorobutyl)benzenesulfonamide

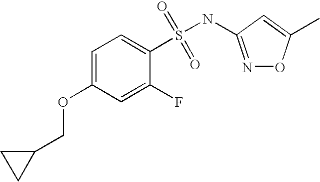

4-(cyclopropylmethoxy)-2-fluoro-N-(5-methylisoxazol-3-yl)benzene-
sulfonamide

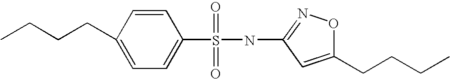

4-butyl-N-(5-butylisoxazol-3-yl)benzenesulfonamide

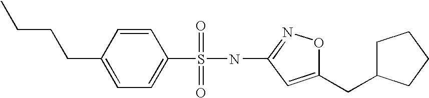

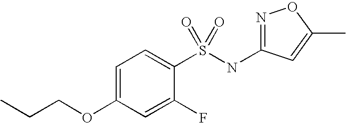

2-fluoro-N-(5-methylisoxazol-3-yl)-4-propoxybenzenesulfonamide

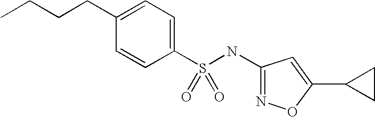

4-butyl-N-(5-cyclopropylisoxazol-3-yl)benzenesulfonamide

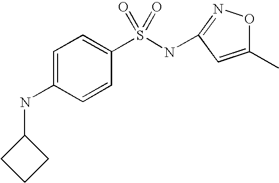

4-(cyclobutylamino)-N-(5-methylisoxazol-3-yl)benzenesulfonamide

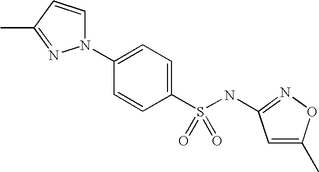

N-(5-methylisoxazol-3-yl)-4-(3-methyl-1H-pyrazol-1-yl)benzene-
sulfonamide

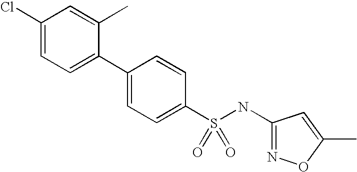

4′-chloro-2′-methyl-N-(5-methylisoxazol-3-yl)-1,1′-biphenyl-4-sulfonamide

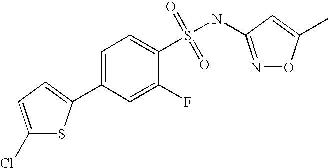

4-(5-chlorothien-2-yl)-2-fluoro-N-(5-methylisoxazol-3-yl)benzene-
sulfonamide

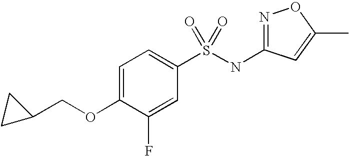

4-(cyclopropylmethoxy)-3-fluoro-N-(5-methylisoxazol-3-yl)benzene-
sulfonamide

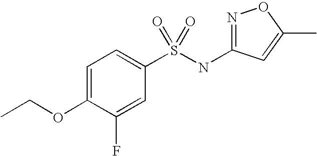

4-ethoxy-3-fluoro-N-(5-methylisoxazol-3-yl)benzenesulfonamide

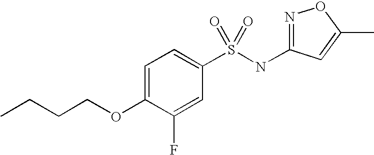

4-butoxy-3-fluoro-N-(5-methylisoxazol-3-yl)benzenesulfonamide

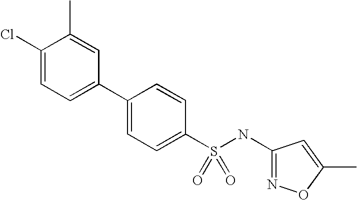

4′-chloro-3′-methyl-N-(5-methylisoxazol-3-yl)-1,1′-biphenyl-4-sulfonamide

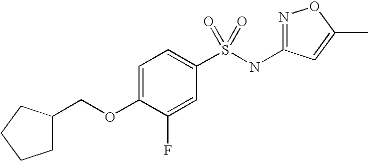

4-(cyclopentylmethoxy)-3-fluoro-N-(5-methylisoxazol-3-yl)benzene-
sulfonamide

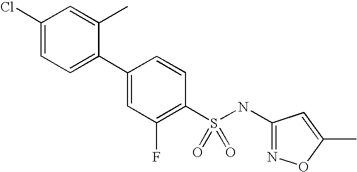

4′-chloro-3-fluoro-2′-methyl-N-(5-methylisoxazol-3-yl)-1,1′-biphenyl-4-
sulfonamide

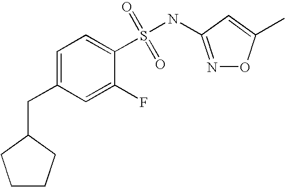

4-(cyclopentylmethyl)-2-fluoro-N-(5-methylisoxazol-3-yl)benzene-
sulfonamide

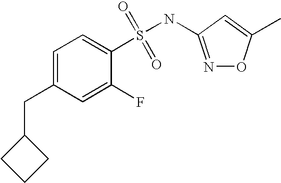

4-(cyclobutylmethyl)-2-fluoro-N-(5-methylisoxazol-3-yl)benzene-
sulfonamide

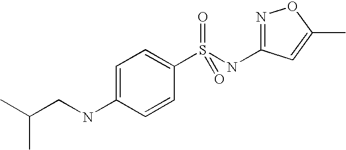

4-(isobutylamino)-N-(5-methylisoxazol-3-yl)benzenesulfonamide

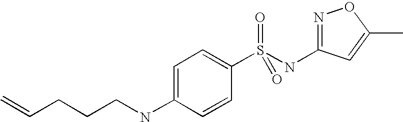

N-(5-methylisoxazol-3-yl)-4-(pent-4-enylamino)benzenesulfonamide

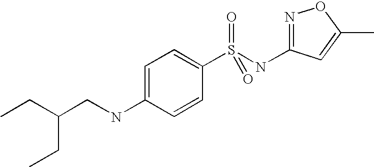

4-[(2-ethylbutyl)amino]-N-(5-methylisoxazol-3-yl)benzenesulfonamide

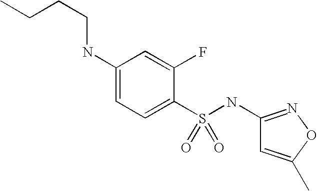

4-(butylamino)-2-fluoro-N-(5-methylisoxazol-3-yl)benzenesulfonamide

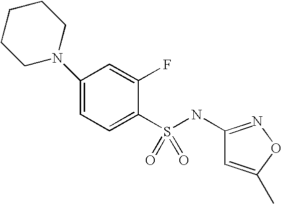

2-fluoro-N-(5-methylisoxazol-3-yl)-4-piperidin-1-ylbenzenesulfonamide

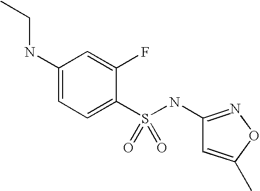

4-(ethylamino)-2-fluoro-N-(5-methylisoxazol-3-yl)benzenesulfonamide

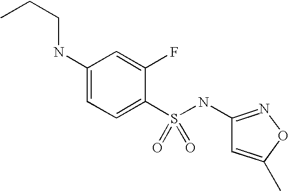

2-fluoro-N-(5-methylisoxazol-3-yl)-4-(propylamino)benzenesulfonamide

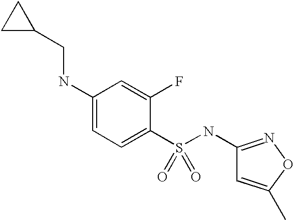

4-[(cyclopropylmethyl)amino]-2-fluoro-N-(5-methylisoxazol-3-yl)benzene-
sulfonamide

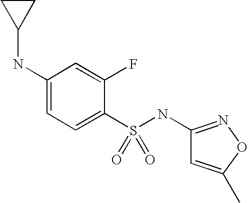

4-(cyclopropylamino)-2-fluoro-N-(5-methylisoxazol-3-yl)benzene-
sulfonamide

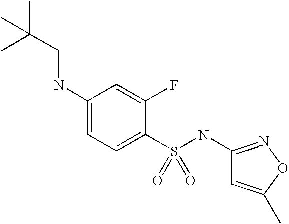

2-fluoro-N-(5-methylisoxazol-3-yl)-4-(neopentylamino)benzene-
sulfonamide

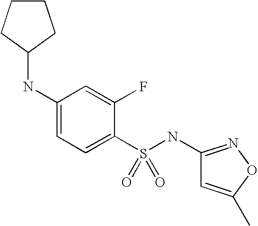

4-(cyclopentylamino)-2-fluoro-N-(5-methylisoxazol-3-yl)benzene-
sulfonamide

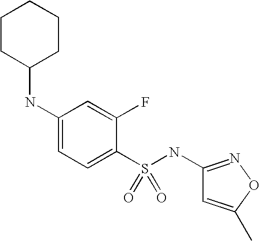

4-(cyclohexylamino)-2-fluoro-N-(5-methylisoxazol-3-yl)benzene-
sulfonamide

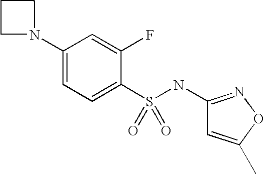

4-azetidin-1-yl-2-fluoro-N-(5-methylisoxazol-3-yl)benzenesulfonamide

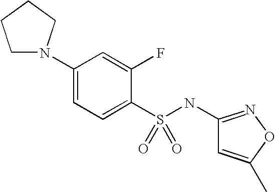

2-fluoro-N-(5-methylisoxazol-3-yl)-4-pyrrolidin-1-ylbenzenesulfonamide

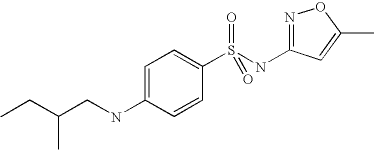

4-[(2-methylbutyl)amino]-N-(5-methylisoxazol-3-yl)benzenesulfonamide

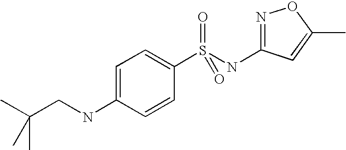

N-(5-methylisoxazol-3-yl)-4-(neopentylamino)benzenesulfonamide

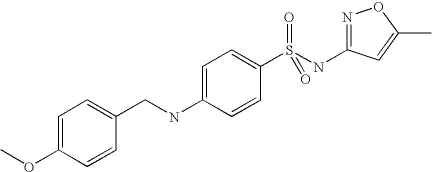

4-[(4-methoxybenzyl)amino]-N-(5-methylisoxazol-3-yl)benzene-
sulfonamide

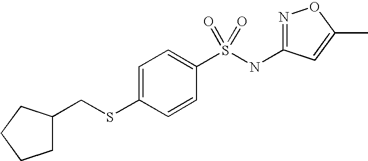

4-[(cyclopentylmethyl)thio]-N-(5-methylisoxazol-3-yl)benzene-
sulfonamide

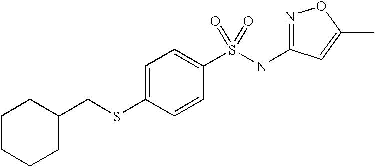

4-[(cyclohexylmethyl)thio]-N-(5-methylisoxazol-3-yl)benzene-
sulfonamide

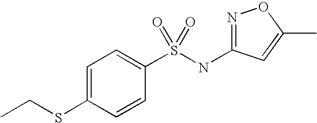

4-(ethylthio)-N-(5-methylisoxazol-3-yl)benzenesulfonamide

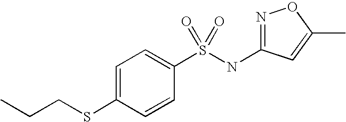

N-(5-methylisoxazol-3-yl)-4-(propylthio)benzenesulfonamide

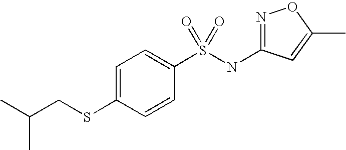

4-(isobutylthio)-N-(5-methylisoxazol-3-yl)benzenesulfonamide

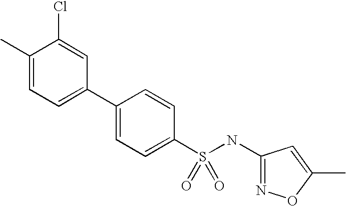

3′-chloro-4′-methyl-N-(5-methylisoxazol-3-yl)-1,1′-biphenyl-4-sulfonamide

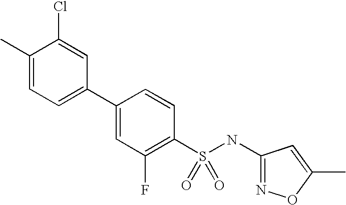

3′-chloro-3-fluoro-4′-methyl-N-(5-methylisoxazol-3-yl)-1,1′-biphenyl-4-
sulfonamide

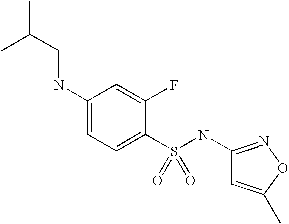

2-fluoro-4-(isobutylamino)-N-(5-methylisoxazol-3-yl)benzenesulfonamide

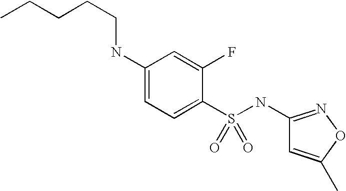

2-fluoro-N-(5-methylisoxazol-3-yl)-4-(pentylamino)benzenesulfonamide

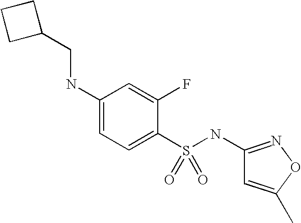

4-[(cyclobutylmethyl)amino]-2-fluoro-N-(5-methylisoxazol-3-yl)benzene-
sulfonamide

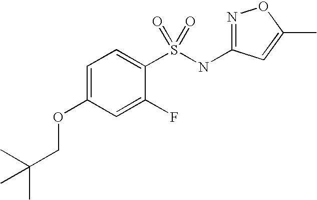

2-fluoro-N-(5-methylisoxazol-3-yl)-4-(neopentyloxy)benzenesulfonamide

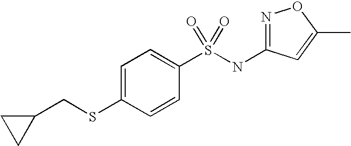

4-[(cyclopropylmethyl)thio]-N-(5-methylisoxazol-3-yl)benzene-
sulfonamide

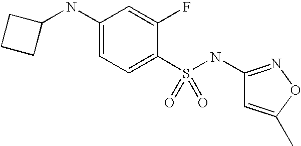

4-(cyclobutylamino)-2-fluoro-N-(5-methylisoxazol-3-yl)benzene-
sulfonamide

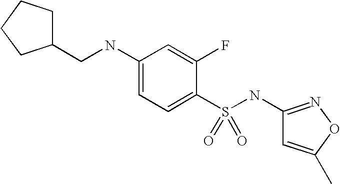

4-[(cyclopentylmethyl)amino]-2-fluoro-N-(5-methylisoxazol-3-yl)benzene-
sulfonamide

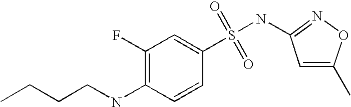

4-(butylamino)-3-fluoro-N-(5-methylisoxazo1-3-yl)benzenesulfonamide

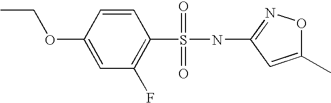

4-ethoxy-2-fluoro-N-(5-methylisoxazol-3-yl)benzenesulfonamide

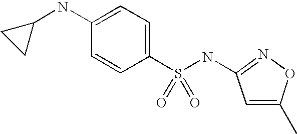

4-(cyclopropylamino)-N-(5-methylisoxazol-3-yl)benzenesulfonamide

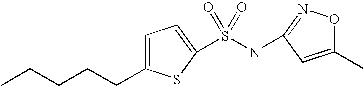

N-(5-methylisoxazol-3-yl)-5-pentylthiophene-2-sulfonamide

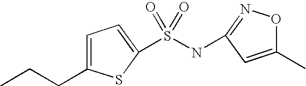

N-(5-methylisoxazol-3-yl)-5-propylthiophene-2-sulfonamide

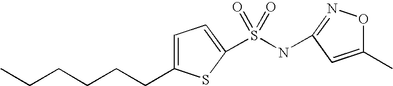

5-hexyl-N-(5-methylisoxazol-3-yl)thiophene-2-sulfonamide

The subject can be a mammal, preferably a human. Identifying a subject in need of such treatment can be in the judgment of a subject or a health care professional and can be subjective (e.g., opinion) or objective (e.g., measurable by a test or diagnostic method).

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents.

The term “mammal” includes mice, rats, cows, sheep, pigs, goats, and horses, monkeys (e.g., cyanmolgus monkeys), dogs, cats, guinae pigs, rabbits, and preferably humans.

The term “halo” or “halogen” refers to any radical of fluorine, chlorine, bromine or iodine.

By “alkyl” is meant cyclic or acyclic alkyl, or combinations thereof, containing the indicated number of carbon atoms. For example, C₁-C₁₂ alkyl indicates that the group may have from 1 to 12 (inclusive) carbon atoms in it. Acyclic alkyl groups may be branched, e.g., tert-butyl, or unbranched, e.g., n-butyl. Cyclic alkyl groups may have one or more rings, which may be bridged or fused. Examples of cyclic alkyl moieties include, but are not limited to, cyclopentyl, norbornyl, decahydronaphthyl, and adamantyl. A combination of an acyclic alkyl and cyclic alkyl may be attached to another moiety through a carbon in either the acyclic or cyclic portion of the group, e.g., —CH₂(C₃H₅) (cyclopropylmethyl). The term “haloalkyl” refers to an alkyl in which one or more hydrogen atoms are replaced by halo, and includes alkyl moieties in which all hydrogens have been replaced by halo (e.g., perfluoroalkyl).

The term “alkoxy” refers to an —O-alkyl radical. Similarly, the alkyl portion of alkoxy substituents may be cyclic, acyclic, or combinations thereof, or branched or unbranched. The cyclic alkyl portion may have one or more rings, which may be bridged or fused.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and having one or more triple bonds. Some examples of a typical alkynyl are ethynyl, 3-hexynyl, and propargyl. One of the triple bond carbons may optionally be the point of attachment of the alkynyl substituent.

The term “aryl” refers to an aromatic monocyclic, bicyclic, or tricyclic hydrocarbon ring system, wherein Any ring atom can be substituted. Examples of aryl moieties include, but are not limited to, phenyl, naphthyl, and anthracenyl.

The term “styryl” refers to a radical of styrene in which the radical may be attached to another moiety through either the a or P carbon of the vinyl group appended to the phenyl ring.

The term “heterocyclyl” or “heterocyclic” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted. The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted. The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3,1-6, or 1-9 heteroatoms of N, O, or S if monocyclic, bicyclic, or tricyclic, respectively). Any ring atom can be substituted.

The term “substituents” refers to a group “substituted” on an acyclic alkyl, cyclic alkyl, or combination thereof; alkenyl; alkynyl; heterocyclyl; heterocycloalkenyl, cycloalkenyl; aryl; or heteroaryl group at any atom of that group. Any atom can be substituted. Suitable substituents include, without limitation, alkyl (e.g., C1, C2, C3, C4, C5 or C6 straight or branched chain alkyl), cycloalkyl, haloalkyl (e.g., C1, C2, C3, C4, C5 or C6 straight or branched chain haloalkyl, e.g., perfluoroalkyl), aryl, heteroaryl, aralkyl, heteroaralkyl, heterocyclyl, alkenyl, alkynyl, cycloalkenyl, heterocycloalkenyl, alkoxy (e.g., C1, C2, C3, C4, C5 or C6 straight or branched chain alkoxy), haloalkoxy (e.g., C1, C2, C3, C4, C5 or C6 straight or branched chain trihaloalkoxy, perfluoroalkoxy), halo, hydroxy, carboxy, carboxylate, cyano, nitro, azido, amino, alkyl amino, acylamino, acylthio, SO₃H, sulfate, phosphate, bivalent oxyalkyloxy (e.g., methylenedioxy, ethylenedioxy), oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

The compounds and methods described herein can be used to treat various fungal mycoses. Mycoses that occur in humans include, without limitation, Actinomycosis, Aspergillosis, Blastomycosis, Candidiasis, Chromomycosis, Coccidioidomycosis, Cryptococcosis, Entomophthoramycosis, Geotrichosis, Histoplasmosis, Mucormycosis, Mycetoma, Nocardiosis, Paracoccidiomycosis, Phaeohyphomycosis, Pneumoscystic pneumonia, Pythiosis, Sporotrichosis, Torulopsosis, Zygomycosis, Chromoblastomycosis, eye infections (e.g., Mycotic keratitis, Endogenous oculomycosis, Extension oculomycosis), Lobomycosis, and Mycetoma. Other syndromes include nail, hair, and skin diseases such as Onychomycosis (Tinea unguium), Piedra, Pityriasis versicolor, Dermatophytosis (e.g., Tinea barbae, Tinea capitis, Tinea corporis, Tinea cruris, Tinea favosa, Tinea imbricata, Tinea manuum, Tinea nigra, Tinea pedis, and Tinea unguium), Dermatomycosis, Otomycosis, Phycomycosis, Phaeohyphomycosis, Rhinosporidiosis, and Trichomycosis. Mycoses affecting animals include, without limitation, Aspergillosis, Candidiasis, Chromomycosis, Cryptococcosis, Dermatophytosis, Entomophthoramycosis, Fungal Keratitis, Mucormycosis, Oomycosis, Pythiosis, and Torulopsosis.

Patients most at risk for fungal infections are those with impairment of neutrophil function due to decreased neutrophil production in the bone marrow, increased neutrophil destruction, or qualitative defects in neutrophil function.

Factors that can cause a decrease in neutrophil production include, but are not limited to (1) administration of cytotoxic drugs, including alkylating agents such as cyclophosphamide, busulfan, and chlorambucil, and antimetabolites such as methotrexate, 6-mercaptopurine and 5-flurocytosine; (2) administration of other drugs known to inhibit neutrophil production including, but not limited to, certain antibiotics, phenothiazines, diuretics, anti-inflammatory agents, and antithyroid drugs; (3) bacterial sepsis infections, viral infections such as HIV, EBV or hepatitis; typhoid, malaria, brucellosis, and tularemia; (4) primary hematologic diseases resulting in bone marrow failure, as well as both hereditary syndromes and acquired defects; (5) bone marrow failure due to tumor invasion or myelofibrosis; and (6) nutritional deficiencies such as deficiency of either vitamin B12 or folate.

Factors that can cause an increase in destruction of neutrophils, thereby rendering an individual susceptible to fungal infections, include, without limitation, the presence of antineutrophil antibodies, autoimmune disease (such as Felty's syndrome, rheumatoid arthritis, or systemic lupus erythematosis), or idiosyncratic reactions to drugs that, in an idiosyncratic way, act as haptens at the surface of neutrophils, initiating immune destruction of neutrophils.

Qualitative defects in neutrophil function that can lead to increased susceptibility to fungal infections include many disease states, for example, leukocyte adhesion deficiency syndromes, neutrophil chemotactic defects, and neutrophil phagocytic and killing defects.

Neutrophil function is also compromised by administration of corticosteroids used in the treatment of a wide variety of diseases. Thus, patients treated with corticosteroids are at increased risk of fungal infections.

Additional factors increasing individual susceptibility to fungal infections include: (1) treatment with broad spectrum antibiotics, especially in the hospital setting and in Intensive Care settings in particular; (2) application of intravenous catheters, particularly central venous catheters; (3) surgical wounds, particularly those associated with intra-abdominal surgeries; (4) bone marrow or solid organ transplantation; (5) cancer chemotherapy; (6) Acquired Immune Deficiency Syndrome; (7) Intensive Care Unit stay; and (8) diabetes. In addition, neonates and aged patients are at increased risk.

The antimicrobial compounds described herein can be used alone or in combination with other antimicrobial compounds, including conventional antimicrobial agents such as known antifungal or antibacterial agents for therapeutic or prophylactic treatment of infection or potential infection. Combination therapies are particularly useful for treatment of infections that exhibit resistance, e.g., acquired or intrinsic resistance, to one or more antimicrobial agents. Thus, combination therapies can be useful for treatment of infection by an organism that exhibits resistance due to either genetic changes or physiological conditions. Combination therapies are also useful in situations where an effective dose of one or more of the agents used in the combination therapy is associated with undesirable toxicity or side effects when not used in combination. This is because a combination therapy can be used to reduce the required dosage or duration of administration of the individual agents. Moreover, the lower dosages often used in a combination therapy may reduce the incidence of acquired resistance to one or more of the agents used in the combination therapy. The individual agents used in combination can act by reducing the growth, replication or viability of a microbe. Moreover, one or more of the individual agents can act by simply reducing the resistance (or increasing the sensitivity) of the microbe to one or more other agents used in the combination.

Among the agents that can be used in combination therapy are polyenes (e.g., Amphotericin B, Mepartricin, Nystatin, Pimaricin, SPA-S-843), candins (e.g., Anidulafungin, Caspofungin, Micofungin, Cilofungin, and V-echinocandin), sordarins (e.g., Azasordarin, GM 222712, and GM 237354), azoles (e.g., Azoline, Albaconazole, Bifonazole, Butoconazole, Clotrimazole, Croconazole, CS-758, Eberconazole, Econazole, Fenticonazole, Fluconazole, Flutrimazole, Fosfluconazole, Isoconazole, Itraconazole, Ketoconazole, Ianoconazole, Miconazole, Neticonazole, Oxiconazole, Posaconazole, PR-2699, Propenidazole, Ravuconazole, Sertaconazole, SSY-726, Sulconazole, Terconazole, Tioconazole, and Voriconazole), allylamines (e.g., Butenafine, Naflifine, Terbinafine), morpholines (e.g., amorolfine), pradimicins (e.g., BMS-181184), and other antifungals (e.g., Alpha interferon, Amantanium bromide, BAY-10-8888, Ciclopirox, Cyclopiroxalamine, DB-289, Exalamide, Flucytosine, Fumagiline, Griseofulvin, Haloprogin, Iseganan, Liranaftate, Natamycin, Nikkomycin, Siccanin, Tolciclate, and Undecylenate).

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include 2 or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The antimicrobial agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, etc.

Antimicrobial agents can be administered, e.g., by intravenous injection, intramuscular injection, subcutaneous injection, or by other routes. They can be injected or otherwise introduced (e.g., via catheter or direct placement) at a site of infection or potential injection. The antimicrobial agents can be administered orally, e.g., as a tablet, gel, paste, slurry, liquid, powder or in some other form. Orally administered compositions can include binders, flavoring agents, and humectants. The agents can be included in dentifrices or oral washes. Thus, oral formulations can include abrasives and foaming agents. The agents can also be administered transdernally or in the form a suppository. They can also be administered in eyedrops.

Antimicrobial agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). Pharmaceutical agents can be sterilized by filter sterilization or by other suitable means.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences. 18th Ed. Mack Publishing Company, 1995.

A prophylactically effective amount of a compound is an amount that, in a given dosage regime, reduces the frequency or severity of infection by a fungal pathogen compared to treatment with a placebo. A therapeutically effective amount of a compound is an amount that, in a given dosage regime, reduces the fungal bioburden within a patient and/or reduces the time to reduce the fungal bioburden to a lower level compared to treatment with a placebo. By “fungal bioburden” is meant the number of fungal cells or spores per unit of sample (e.g., the number of cells or spores per gram of tissue). The number of cells can be determined by methods including, but not limited to, calculation of fungal biomass, PCR signal with fungal-specific primers, hybridization, histologic examination, and plating for colony forming units. Specific methods can be more or less applicable depending on the characteristics of the organism and the treatment. Therapeutically effective doses can be determined using an animal model or via clinical studies. Experimental animals suffering from a microbial infection are often used to determine an initial therapeutic regime that can be further verified in human clinical trials.

The antimicrobial agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

For agricultural uses, the compositions or agents identified using the methods disclosed herein may be used as chemicals applied as sprays or dusts on the foliage of plants, or in irrigation systems. Typically, such agents are to be administered on the surface of the plant in advance of the pathogen in order to prevent infection. Seeds, bulbs, roots, tubers, and corms are also treated to prevent pathogenic attack after planting by controlling pathogens carried on them or existing in the soil at the planting site. Soil to be planted with vegetables, ornamentals, shrubs, or trees can also be treated for control of a variety of microbial pathogens. Treatment is preferably done several days or weeks before planting. The chemicals can be applied by either a mechanized route, e.g., a tractor, cropduster, spraying, or with hand applications. In addition, chemicals identified using the methods of the assay can be used as disinfectants.

In addition, the compounds described herein can be coated onto or integrated into materials used to make catheters, including but not limited to intravenous, urinary, intraperitoneal, ventricular, spinal and surgical drainage catheters, in order to prevent colonization and systemic seeding by potential pathogens. Similarly, the compounds described herein may be added to the materials that constitute various surgical prostheses and to dentures to prevent colonization by pathogens and thereby prevent more serious invasive infection or systemic seeding by pathogens.

The details of one or more embodiments of the invention are set forth in the accompanying drawings and the description below. Other features, objects, and advantages of the invention will be apparent from the description below.

### BRIEF DESCRIPTION OF THE DRAWINGS

FIG. 1 is a scheme showing the numbers of lesions on mice kidneys when treated with compounds described herein.

FIG. 2 is a graph showing the histology scores of mice treated with compounds described herein.

### DETAILED DESCRIPTION

The compounds described herein can be synthesized according to the procedures shown below, where substituents R1-R81 are as described above.

Treatment of representative sulfonylchlorides (1) with 3-amino-5-alkylisoxazole (2) in the presence of base provides the desired isoxazole benzenesulfonamides (3) in good to very good yields (Eq. 1).

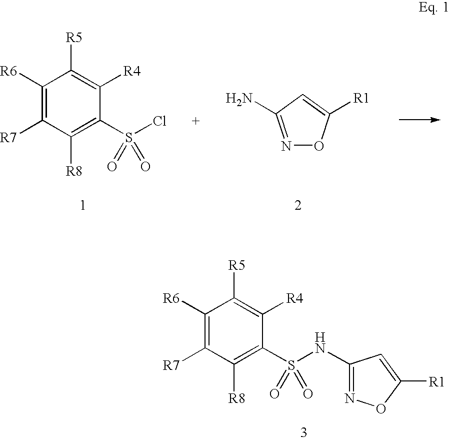

When R⁶ is a leaving group (e.g., halo, triflate, mesylate, nosylate, etc.), it is possible to replace the leaving group with another substituent.

For example, exposure of, e.g., 3 (R⁶=I or Br) to an aryl boronic acid, an aryl stannane, or an aryl zinc halide, in the presence of a palladium catalyst [e.g., Pd(PPh₃)₄ or PdCl₂(dppf)] can result in the production of biphenyl derivatives. The reaction may be carried out in the presence of a base (e.g., K₂CO₃ or triethylamine). The transformation may also be conducted with heteroaromatic coupling partners (e.g., thiophene, pyridine, furan, etc.) bearing boronic acid, trialkyltin, and halozinc substituents. Metal catalyzed coupling reactions are described in: Herrmann, Wolfgang A. The Suzuki cross-coupling. Applied Homogeneous Catalysis with Organometallic Compounds (2nd Edition) (2002), 1 591-598 (boronic acid cross couplings); Hassan, Jwanro; Sevignon, Marc; Gozzi, Christel; Schulz, Emmanuelle; Lemaire, Marc. Aryl-Aryl Bond Formation One Century after the Discovery of the Ullmann Reaction. Chemical Reviews (2002), 102(5), 1359-1469 (trialkyltin cross couplings); and Negishi, Ei-Ichi; Liu, Fang. Palladium- or nickel-catalyzed cross-coupling with organometals containing zinc, magnesium, aluminum, and zirconium. Metal-Catalyzed Cross-Coupling Reactions (1998), 1-47 (organozinc cross couplings).

Palladium-catalyzed coupling of e.g., 3, with an amine, an alkyne, an E-vinyl borinate ester, or a trialkyl borane can afford compounds containing an amino group, an alkynyl group, an alkenyl group, and an alkyl group respectively at R⁶. These coupling reactions are described in M. H. Ali, S. L. Buchwald, J. Org. Chem. 2001, 66, 2560-2565, and J. P. Wolfe, H. Tomori, J. P. Sadighi, J. Yin, S. L. Buchwald J. Org. Chem. 2000, 65, 1158-1174 (amines); W. G. B. van Henegouwen, R. M. Fieseler, F. P. J. T. Rutjes, H. Hiemstra, Angew. Chem. Int. Ed. Engl. 1999, 38, 2214, and G. Esteban, M. A. Lopez-Sanchez. M. E. Martinez, J. Plumet Tetrahedron 1998, 54, 197 (vinyl borinates); N. Miyaura et al. Tetrahedron Lett. 1986, 27, 6369, and S. R. Chemler, D. Trauner, S. J. Danishefsky Angew. Chem. Int. Ed. Engl. 2001, 40, 4544 (trialkylboranes); K. Songashira, Y. Tohda, N. Hagihira Tetrahedron Lett. 1975, 4467-70, and S. Thorand, N. Krause J. Org. Chem. 1998, 8551 (alkynes). The alkynylated compounds can subsequently be hydrogenated with a reduced activity catalyst, e.g., Lindlar's catalyst, to afford the corresponding Z-olefins. This two-step Z-olefin synthesis is complementary to the vinyl borinate coupling above, which yields E-olefins.

Treatment of 4-bromo or 4-iodo substituted derivatives of 3 (R6=Br, I) with an arylboronic acid (4) in the presence of a base and a palladium catalyst (the Suzuki coupling

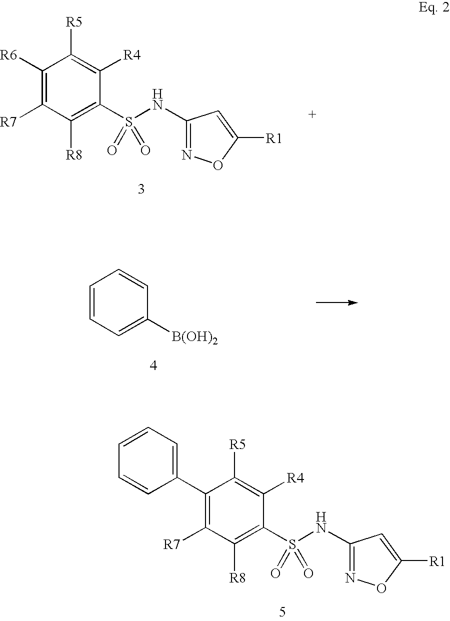

reaction) affords biphenyl compounds such as (5) in good yields (Eq. 2). The coupling of 4-bromo or 4-iodo substituted derivatives of 3 (R⁶=Br, I) is not limited to arylboronic acids like 4, but may also include heteroaromatic boronic acids such as thiopheneboronic acids, pyridineboronic acids, furanboronic acids, and the like. In addition, 4-bromo or 4-iodo substituted derivatives of 3 (R⁶=Br, I) can also be coupled to aryl and heteroaryl stannyl derivatives in the presence of palladium catalysts.

Treatment of 4-bromo or 4-iodo substituted derivatives of 3 (R6=Br, I) with a secondary amine (6) in the presence of base and a palladium catalyst, using Hartwig or Buchwald conditions, affords isoxazole amino-benzenesulfonamide compounds such as (7) in moderate to good yields (Eq. 3).

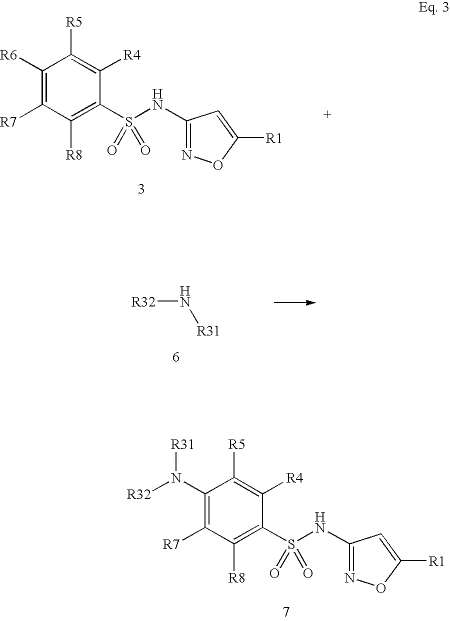

Treatment of 4-bromo or 4-iodo substituted derivatives of 3 (R6=Br, I) with an acetylene derivative (8) in the presence of a base and a palladium catalyst, the Sonogoshira reaction, affords acetylene substituted compounds such as (9) in good yield (Eq. 4). The resulting acetylene substituted compounds, (9), can then be hydrogenated with a reduced activity catalyst, such as Lindlar's catalyst, to afford the corresponding Z-olefins.

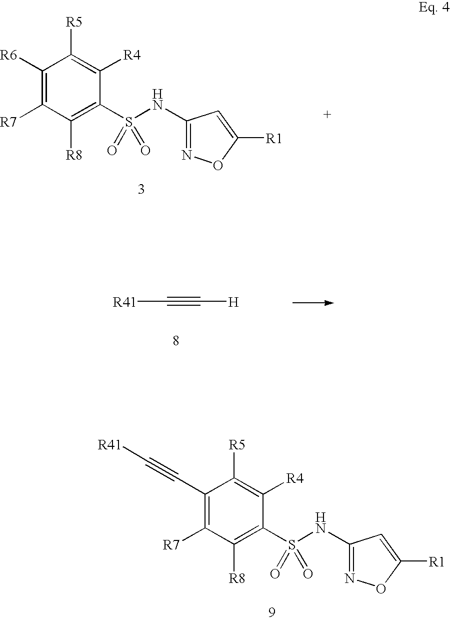

Treatment of a 4-fluorosubstituted derivative of 3 (R6=F) with an alcohol, phenol, thiol, or thiophenol (10) in the presence of base affords the alkoxy (11, X=O) or thioalkoxy (11, X=S) substituted compounds such as (11) in good to excellent yields (Eq. 5). The latter species can be generated in situ from the corresponding alcohols, phenols, thiols or thiophenol (e.g., R¹⁴XH) with a base e.g., sodium hydride, potassium hydride, potassium hydroxide, or a tertiary amine.

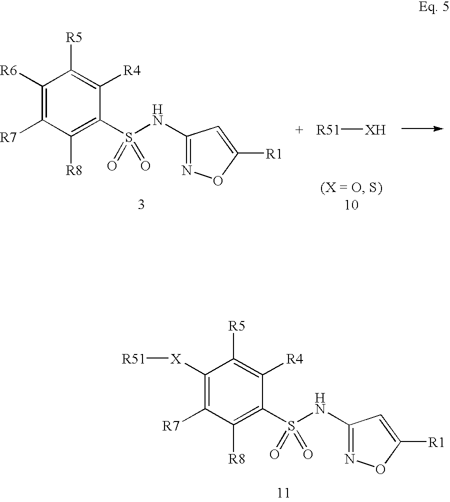

Treatment of a 4-amino derivative of 3 (R6=NH2) with an aldehyde or ketone in the presence of a reducing agent such as sodium triacetoxyborohydride affords the corresponding alkylamino substituted compounds such as (12) in good yield (Eq. 6).

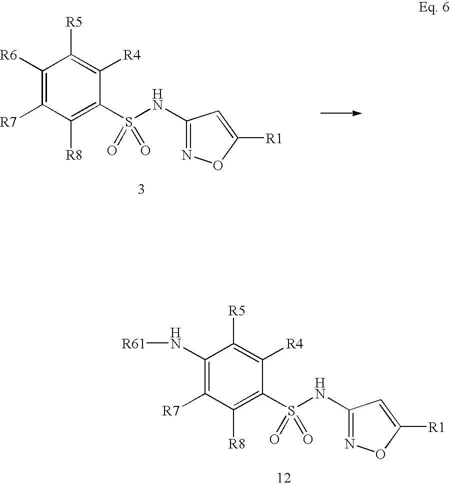

Vinyl borinate esters, such as the E-catechol borane (13) can undergo coupling with 4-bromo or 4-iodo substituted derivatives of 3 (R6=Br, I) to provide the corresponding substituted E-olefins such as 14 (Eq. 7).

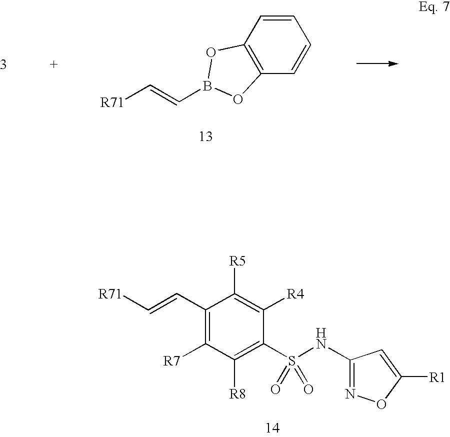

Trialkylboranes are commercially available or can be prepared from the reaction of an olefin with 9-borabicyclo[3.3.1]nonane (9-BBN). Trialkylboranes can be coupled with 4-bromo or 4-iodo substituted derivatives of 3 (R6=Br, I) to provide 4-alkyl derivatives such as 15 (Eq. 8).

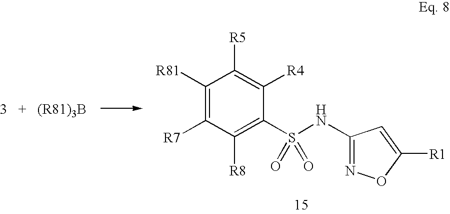

The compounds described herein have been prepared by the coupling of a substituted sulfonyl chloride (1) with an amine (2) as illustrated in Eq. 1. Sulfonyl chlorides in turn have been prepared by a number of different methods including the direct chlorosulfonation of an aromatic compound such as 16 (X=H) (see Eq. 9), in the presence of excess chlorosulfonic acid. This method has also been used for the preparation of heterocyclic chlorosulfonyl derivatives, such as 18, from the corresponding heterocycle 17, X═H (see Eq. 10). Treatment of bromo aromatic derivatives of 16 (X=Br) with n-butyllithium, followed by sulfur dioxide, and then sulfuryl chloride, is another method for the preparation of the aromatic chlorosulfonyl (1) and heteroaromatic chlorosulfonyl (18). There are still other methods for the preparation of aromatic chlorosulfonyl derivatives (1) and heteroaromatic chlorosulfonyl derivatives (18) that are known in the art and could be employed for the preparation of this component of the compounds described herein.

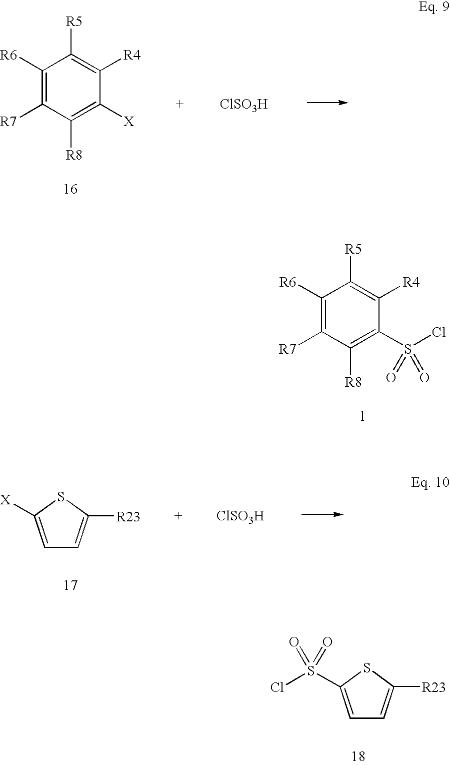

The compounds described herein have also been prepared by the palladium catalyzed coupling of aryl zinc and alkyl zinc derivatives (19) with 4-bromo or 4-iodo substituted derivatives of 3 (R6=Br, I) to provide biaryl (5) and alkyl substituted (15) compounds, respectively, in good to excellent yield (Eq. 11).

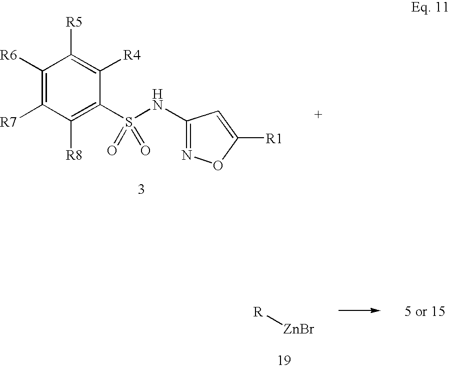

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Fungal inhibiting compounds can be identified through both in vitro (cell and non-cell based) and in vivo methods. A description of these methods is described in the Examples.

The following examples are meant to be illustrative, and are not meant to limit the invention described herein.

### EXAMPLE 1

### Synthesis of N-(5-methyl-3-isoxazolyl)-4′-(3,5-difluorophenyl)benzensulfonamide

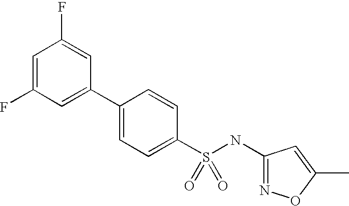

A 25 mL round bottomed flask equipped with a nitrogen inlet, provisions for magnetic stirring and a reflux condenser was charged with Pd(PPh₃)₄ (0.01 mmol), N-(5-methyl-3-isoxazolyl)-4-bromobenzensulfonamide (0.2 mmol), aqueous K₂CO₃ (0.2 mL, 2M), and toluene (1 mL). The solution was treated with 3,5-difluorophenylboronic acid (0.28 mmol) in ethanol (0.6 mL) and heated to reflux overnight with vigorous stirring. The reaction mixture was diluted with 1 mL of H₂O, and the solution was extracted three times with ethyl acetate. The combined extracts were washed with brine, dried over anhydrous Na₂SO₄, filtered, and concentrated in vacuo to afford a solid. The crude solid was recrystallized from ethyl acetate/hexanes to provide 56 mg, 80% of N-(5-methyl-3-isoxazolyl)-4′-(3,5-difluorophenyl)benzensulfonamide, mp 175° C. Rf=0.65, silica gel, 1:1 ethyl acetate/hexanes. ¹H NMR (CDCl₃) 9.60(s, 1H), 7.95(d, 2H), 7.65(d, 2H), 7.10(m, 2H), 6.85(m, 1H), 6.60(s, 1H) 2.80(s, 3H). LC-MS M-H=349.

### EXAMPLE 2

### Synthesis of 5-Butyl-N-(5-methylisoxazol-3yl)thiophene-2-sulfonamide

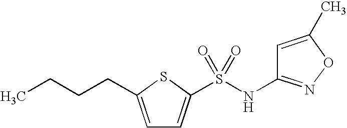

A solution of 5-butylthiophene-2-sulfonyl chloride (3.7 g, 15.5 mmol) (Chemical Abstracts Registry Number 81566-65-6; prepared by the method described in Siedel, W.; Sturm, K. (Farbwerke Hoechst A.-G.) Application DE 19581022; Chemical Abstracts Number 56:2349, 1962) in pyridine (20 mL) was treated with 3-amino-5-methylisoxazole (1.5 g, 15.3 mmol) and stirred at ambient temperature for 12 h. The solution was concentrated in vacuo and the residue taken up in ethyl acetate. The ethyl acetate solution was washed with 1N hydrochloric acid, water, brine, dried over anhydrous sodium sulfate, and concentrated in vacuo. The residue, 4.8 g, was then purified by flash chromatography over silica gel with hexanes/ethyl acetate to afford 3.3 g, 71% of 5-butyl-N-(5-methylisoxazol-3yl)thiophene-2-sulfonamide, mp 96.5-100.7° C. Rf=0.66, silica gel, 1:1 ethyl acetate/hexanes. LC-MS M-H=299.

### EXAMPLE 3

### Preparation of 4-(cyclopentyl)-benzenesulfonamid (5-methylisoxazole-3-yl)

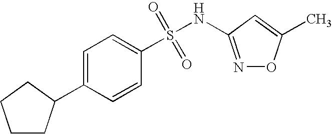

4-bromobenzenesulfonamid-isoxazole (500 mg, 1.57 mmol) and palladium tetrakis triphenylphosphine (95 mg, 0.08 mmol) were dissolved in 6.5 mL dry DMF. Cyclopentylzinc bromide (0.5 M in THF, 6.80 mL, 3.40 mmol) was added via syringe at room temperature. The brownish-green solution was stirred for 2 h at 85° C. until analysis by LC-MS indicated complete consumption of the starting material. The reaction mixture was cooled to room temperature, quenched with 20 mL of sat. NH₄Cl and brought to pH=2-4 with 1N HCl. The mixture was extracted with 3×50 mL of dichloromethane; the combined organic layers were washed with 30 mL brine, dried over Na₂SO₄ and concentrated in vacuo. Purification of the crude product by flash column chromatography (hexanes:ethyl acetate 2:1) followed by recrystallization from hexanes: diethyl ether 3:1 gave pure product (293 mg, 61%) as a white solid.

¹H NMR (CDCl₃):; δ 7.76 (d, 2H, J=8.7 Hz,), 7.36 (d, 2H, J=8.4 Hz,), 6.27 (m, 1H), 3.11-2.98 (m, 1H), 2.38 (d, 3H, J=0.9 Hz ), 2.16-2.05 (m, 2H), 1.90-1.49 (m, 6H). LRMS m/z 306 (M−H+); m.p. 157.5° C.

### EXAMPLE 4

### Identification of an Inhibitor of Fungal Invasion

A screen was conducted to identify candidate inhibitors of fungal invasion. The initial screen entailed testing the ability of a test compound to inhibit expression of a reporter gene (HWP1-lacZ). HWP1 is a C. albicans gene whose expression has been correlated with the hyphal transition. Inhibition of HWP1 expression is expected to correlate with inhibition of fungal invasion. Stationary phase Candida albicans cells were used in the initial screen. This assay identified a compound, Compound I (below), as an effective inhibitor of invasion of stationary phase C. albicans.

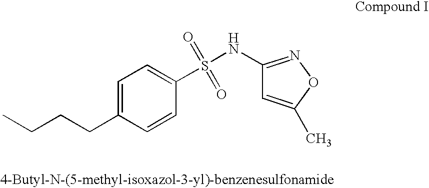

Compound I inhibits invasion of stationary phase C. albicans by 50% at 0.85 μg/mL as determined by the stationary phase C. albicans anti-invasion assay described below. Additional testing determined that Compound I inhibits invasion of logarithmic phase C. albicans by 50% at 0.0049 μg/mL as determined by the logarithmic phase C. albicans anti-invasion assay described below.

Stationary Phase C. albicans Anti-Invasion Assay

Stock cultures for use in this assay were prepared by streaking C albicans (MC295: ura3Δ::imm434/ura3Δ::imm434 his1::hisG/his1::hisG arg4::hisG/arg4::hisG his1::hisG/+HWP1/HWP1p-lacZ(URA3) gal::ARG41GAL) cells for isolation on a YPD agar plate. The cells were grown at 30° C. for 14-18 h, and an isolated colony was added to 5 mL of YPD broth and grown on a roller drum (60 rpm) for 14-18 h at 30° C. Stocks were prepared by aliquotting 600 μL of culture into 1 mL of 25% glycerol. The stocks were stored at −80° C.

Cultures for assays were prepared by streaking frozen culture stock on a YPD agar plate and growing the cells for 30° C. for 14-18 h. A single colony was inoculated into 5 mL of YPD in a test tube and grown on a roller drum (60 rpm) for 2 days at 30° C. to late stationary phase (OD₆₀₀˜30). For the assay, the primary cell suspension stock was diluted to an OD₆₀₀ of 1.0 (˜2×10⁷ cells/mL) to make a 10× stock.

To prepare an assay culture, 10 μL of 10× cell stock was added to 80 μL of Inducing Media (see below) and 10 μL of 10× test compound stock or, as a control, DMSO in a Coming, tissue-culture treated, flat-bottom, microtiter plate. The plate was incubated at 37° C. for 3 h. To measure invasion activity, β-galactosidase activity was determined using MUG. Briefly, MUG stock was deleted to 0.4 mg/mL in Z buffer to create MUG/Z solution (see below). To initiate the reaction 100 μL of MUG/Z solution was added to each test well (final MUG concentration of 0.2 mg/mL) and the plate was incubated at 22° C. for 1 h. The reaction was quenched with 60 μL of 1 M sodium bicarbonate and fluorescence was measured using a Spectromax Gemini Fluorometer (Excitation 360 nm, Emission 449 nm). Inhibition of invasion was calculated using the formula: % inhibition=(1-((unknown)ₐᵥₑ−(positive drug control)ₐᵥₑ)/((no drug control)ₐᵥₑ−(positive drug control)ₐᵥₑ))*100.

Logarithmic Phase C. albicans Anti-Invasion Assay

Stock cultures for use in this assay were prepared by streaking C. albicans (MC295) cells on a YPD (Yeast Extract Peptone Dextrose) agar plate. The cells were grown at 30° C. for 14-18 h and an isolated colony was picked and inoculated into a 250 mL Erlenmeyer flask containing Non-Inducing Medium (see below) that had been sterilized by passing it through a 0.22 μm filter. The flask was placed on a rotary shaker at 30° C. between 200-250 rpm, for 14-18 h. The optical density at 600 nm (OD₆₀₀) was determined using non-inducing media as a blank. The overnight culture was diluted with 15% glycerol to a final OD₆₀₀ of 0.1 and aliquotted into 1 mL sterile cryonic tubes that were capped and stored at −80° C.

To prepare assay cultures, approximately 350 μL of thawed Candida albicans (MC295) containing HWP1-lacZ stock is was inoculated into a flask containing 50 mL of NI medium. In addition, 1/1 (vol/vol) serial dilutions were made into two additional flasks each containing 25 mL of NI medium. The flasks were placed on a rotary shaker at 30° C. between 200-250 rpm, for 14-18 h. The OD₆₀₀ was determined using NI medium as a blank. The flask containing cells at an OD₆₀₀ of 0.8-1.0 was used as a 10× stock for the log anti-invasion assay.

To prepare an assay culture, 10 μL of 10× cell stock was added to 80 μL of Inducing Media (see below) and 10 μL of 10× test compound stock or, as a control, DMSO in a Coming, tissue-culture treated, flat-bottom, microtiter plate. The plate was incubated at 37° C. for 3 h. To measure invasion activity, β-galactosidase activity was determined using MUG. Briefly, MUG stock was deleted to 0.4 mg/mL in Z buffer to create MUG/Z solution (see below). To initiate the reaction 100 μL of MUG/Z solution was added to each test well (final MUG concentration of 0.2 mg/mL) and the plate was incubated at 22° C. for 1 h. The reaction was quenched with 60 μL of 1 M sodium bicarbonate and fluorescence was measured using a Spectromax Gemini Fluorometer (Excitation 360 nm, Emission 449 run). Inhibition of invasion was calculated using the formula: % inhibition=(1-((unknown)ₐᵥₑ−(positive drug control)ₐᵥₑ)/((no drug control)ₐᵥₑ−(positive drug control)ₐᵥₑ))*100.

Preparation of MUG stocks: To prepare a MUG stock 4-methylumbelliferyl beta-D-galactoside (MUG, Sigma # M1633) was diluted in 100% DMSO to a final concentration of 54 mg/mL and stored at −20° C. in cryovials. New stocks of MUG were prepared every 3 months.

Preparation of Inducing Media: To prepare this media, 100 mL of YNB 2× salts (1.5 g Yeast Nitrogen base (w/o amino acids or ammonium sulfate), 5 g Ammonium Sulfate, 0.2 mM Inositol was combined with 60 mL of 40% Glucose, 60 mL of 1M MOPS buffer, and 230 mL of deionized water to bring the total volume to 950 mL. The pH was adjusted to 7.5 using 1N NaOH. The volume was brought to 1.0 L with deionized water and the media was sterilized by passing it through a 0.2 micron filter. The media was preferably stored protected from light.

The anti-invasion activity of Compound I was further investigated using a C. albicans morphology assay. In this assay, described in greater detail below, the morphology of cells was assessed and scored on a 1-5 scale. A score of 1 was assigned to non-hyphal cells, “yeast-like” cells and budding cells, and a score of 5 was assigned to cells having many, relatively long (wild-type) hyphae. In this assay, the cells in cultures exposed to 10 μM Compound I had an average score of 1. This score indicates that Compound I is highly effective in inhibiting fungal invasion.

Morphology Assay

A frozen stock culture was prepared by streaking C. albicans (MC303: ura3 Δ:: imm434/ura3Δ:: imm434 arg4::hisG/arg4::hisG ade2:: URA3:.pTEF1-lacZ/ADE2 hisl::hisG/HIS1 gal1::ARG4/GAL1) cells on a YPD agar plate and grown at 30° C. for 14-18 h. An isolated colony was added to 5 mL of YPD broth and grown on a roller drum (60 rpm) for 14-18 h at 30° C. Next, 600 μL of culture were aliquotted into 1 mL of 25% glycerol, and the tubes are stored at −80° C.

Frozen MC303 C. albicans cell stock was struck for isolation of a single colony using a sterile loop on a YPD agar plate and grown at 30° C. for 14-18 h. A single colony was inoculated into 5 mL of YPD in a test tube and grown on a roller drum (60 rpm) for 2 days at 30° C. to late stationary phase (OD₆₀₀˜30). For the assay, this stock was diluted to 2.5×10⁵ cells/mL to make a 10× stock. Next, 10 μL of this cell stock was added to 80 μL of Inducing Media and 10 μL of 10× compound stock or DMSO in a Coming, tissue-culture treated, flat-bottom, microtiter plate. The plate was incubated at 37° C. for 24 h. Next, 10 μL of formaldehyde (37%) was added to each well and the cells were fixed for 10 min. The liquid was gently decanted from the plate and each well was examined under a microscope. Each well was photographed and observed cells are scored on a scale of 1-5. A 5 indicates that C. albicans cells have many long hyphae and is the wild type (WT) phenotype (no drug control). A 1 indicates cells are non-hyphal with only “yeast-like” cells and budding cells.

Scores of 2, 3, and 4 indicate shorter length or reduced quantity of hyphae when compared with WT (2 being very near non-hyphal and 4 being close to WT).

Mammalian Cell Toxicity Assay

To investigate the toxicity of Compound I for mammalian cells, human hepatoma cell line HepG2 were exposed to Compound I and the (LD₅₀) was determined. Briefly, HepG2 human hepatoma cells (American Type Culture Collection, Bethesda Md.) were plated at 1×10⁵ cells/well in tissue culture treated 96 well plates and incubated at 5% CO₂, 37° C. for 18 h prior to initiation of the assay. The compound stocks at 100 mM in DMSO were added to DM (defined media, media without serum with added insulin, selenium and transferrin) at an initial concentration of 1000 μM and serially diluted 1 to 3 in DM in a 96 well plate. For 20 μL of a test solution, 3.5 μL sample is added to 346.5 μL media for an initial concentration of 1000 μM. These dilutions are added to the cells at final sample concentrations between 0.5 and 1000 μM (<1% DMSO). Controls included: media only (negative control) and 0.1% Triton-X (positive control). Control drugs (tamoxifen and 2-thiouracil) were also used to verify each assay. The samples were incubated at 37° C. in humidified 5% CO₂ atmosphere for 4 h.

Next, sterile Alamar Blue solution (final 0.5% w/v) was added to each well and the cells were incubated at 37° C. in 5% CO₂ for at least 3 h. The plate was read directly on the Tecan Spectrafluor Plus reader in the fluorescent mode at excitation 530 nm and emission 595 nm. The blank was subtracted from the total fluorescence to give the net fluorescence for that well. This total was compared to the control in the absence of the compound. An LD₅₀ (concentration at 50% of lethal dose) is calculated as the concentration that leads to a response of 50% compared to the control cells. Thus, cytotoxicity is measured as percent of inhibition of cell viability as determined by the Alamar Blue assay. The expected LD₅₀ ranges of the two control drugs are as follows: tamoxifen, LD₅₀=26.9 1M and 2-thiouracil, LD₅₀>1000 μM.

For Compound I the mammalian cell LD₅₀ was determined to be greater than 1000 μM.

### Example 5

### Generation and Testing of Additional Inhibitors of Fungal Invasion

Based on Compound I, a number of structurally related compounds were prepared and tested for the ability to inhibit fungal invasion. These compounds are depicted in the Table. In this Table the structure of the tested compound in depicted in the first column. Results are reported for the following tests: C. albicans logarithmic phase growth invasion assay (column 2), C. albicans stationary phase growth invasion assay (column 3), C. albicans invasion morphology phenotype upon exposure to the test compound at 10 μM (column 4), and mammalian cell toxicity (column 5).

### Example 6

### Effectiveness of Inhibitors of Fungal Invasion In Vivo

Compound II, depicted below, was found to be an inhibitor of fungal invasion (see Table).

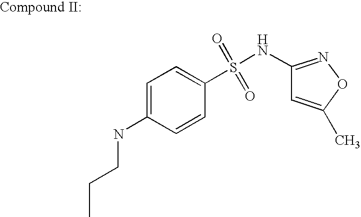

A mouse model of fungal invasion was used to examine the in vivo efficacy of Compound II in reducing invasion by C. albicans.

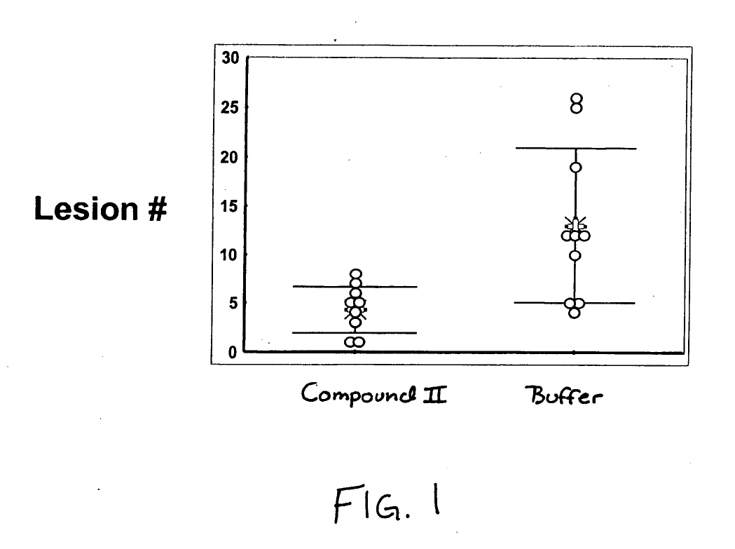

Briefly, 30 min prior to infection (t=−30 min) 1 mg of Compound II in buffer (10 treatment mice) or buffer only (10 control mice) was administered IP. At t=0 all mice were inoculated with 2×10⁶ C. albicans. At t=6 h and t=14 h the IP treatment with Compound II or buffer was repeated. In addition, at t=2, t=4 h, t=10 h, and t=18 h 1 mg of Compound II or buffer only was administered orally. The mice were sacrificed at about t=19 h and the kidneys of the mice were examined for histologic signs of fungal invasion by counting the number of lesions. The results of this analysis are presented in FIG. 1 where it can be seen that treatment with Compound II decreases the number of lesion relative to the control.

Compound III, depicted below, was also found to be an inhibitor of fungal invasion (see Table).

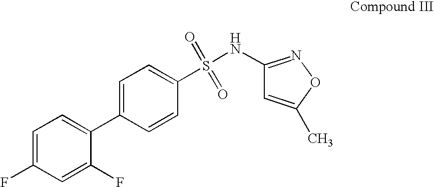

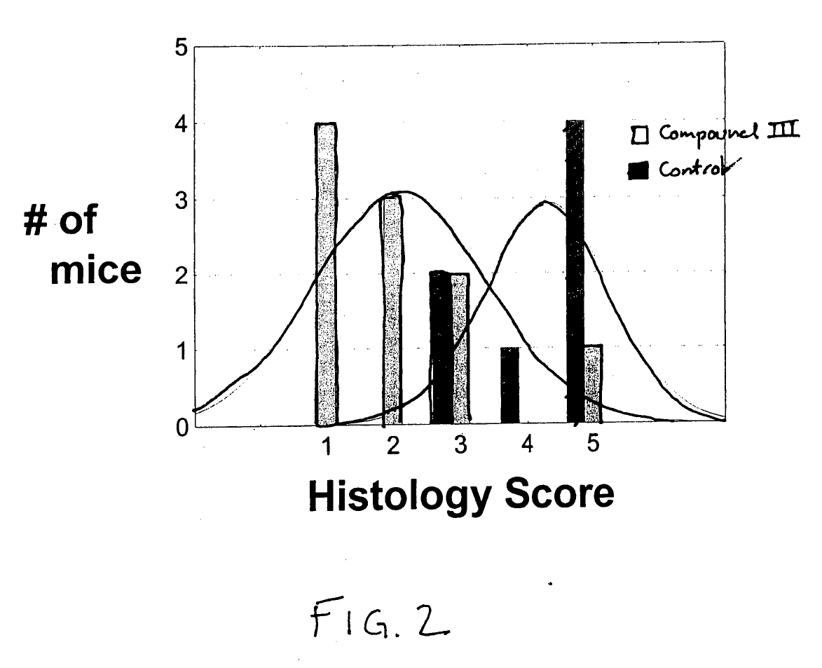

A mouse model of fungal invasion was used to examine the in vivo efficacy of orally administered Compound III in reducing invasion by C. albicans. Briefly, one day prior to infection (t=−24 h) mice were switched to a powdered diet supplemented Compound III (treatment mice) or no supplement (control mice). This diet was continued until the mice were sacrificed at t=48 h. The kidneys of the mice were then examined for histologic signs of fungal invasion. In the scoring system used, a score of 5 indicates the presence of many, large fungus-dominated lesions, a score of 3 indicates the presence of many inflammatory lesion and fungus-dominated lesion of mixed size, and a score of 1 indicates few, mainly inflammatory lesions. The results of this analysis are presented in FIG. 2 where it can be seen that oral treatment with Compound III decreases severity of fungal invasion relative to the control.

Data for selected compounds is summarized in Table 2.

| | | | C. albicans | | |
|---|---|---|---|---|---|
| | | | Overnight | | AFDD |
| | C. albicans | | Growth | C. albicans | Mammalian |
| | LOG IC50 | C. albicans STAT | Inhibition | Phenotype | Cytotoxicity |
| MOLSTRUCTURE | (uM) | IC50 (uM) | (%) | Rating | LD50 (uM) |
| 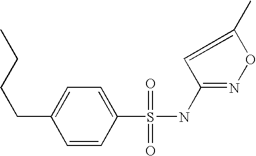 | 0.00603 | 0.8975 | 19.4 | 1, 1*, 2* | >1000 |
| 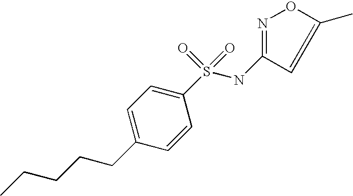 | 0.00928 | 1.409 | −49.4 | 4 | |
| 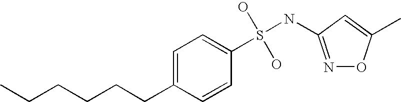 | 0.0099 | 2.865 | 32.5 | 2, 4, 5 | 449.1 |
| 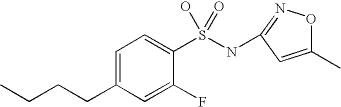 | 0.01 | >20 | 3.8 | 5 | |
| 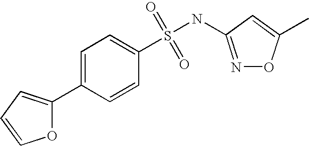 | 0.039 | >20 | 23.9 | 4 | >1000 |
| 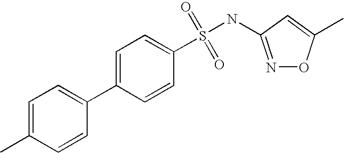 | 0.041 | >20 | 12.3 | 5, 1*, | >1000 |
| 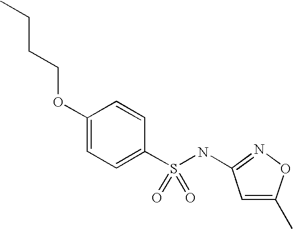 | 0.042 | 11.731 | −6.4 | 2, 5, 1* | >1000 |
| 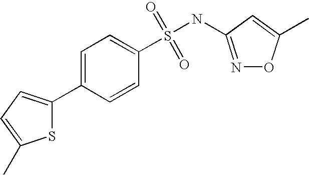 | 0.05 | 4.905 | 22.4 | 5, 1* | |
| 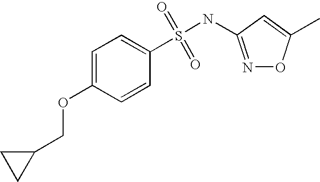 | 0.051 | 11.47 | −7.3 | 5 | |
| 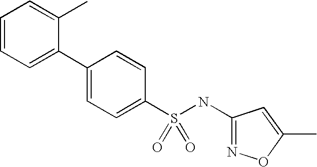 | 0.056 | >20 | 4.8 | 5 | |
| 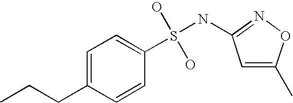 | 0.077 | >20 | 2.0 | 5 | |
| 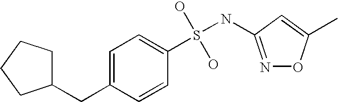 | 0.07925 | 4.469 | 0.9 | 2, 4, 5 | 660.9 |
| 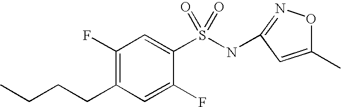 | 0.087 | >20 | −14.5 | 5 | |
| 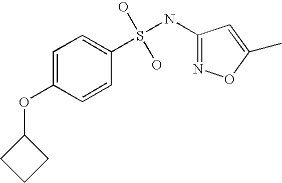 | 0.088 | 8.44 | −5.0 | 5 | |
| 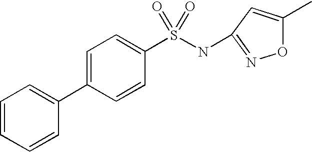 | 0.0893 | 3.606, >20 | 19.5 | 2, 5, | >1000 |
| 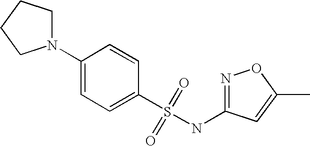 | 0.091 | 3.7285 | 4.4 | 4, 5 | |
| 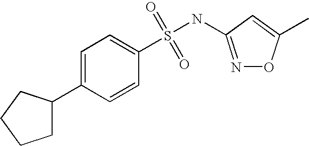 | 0.11 | 19.8 | 13.9 | 5 | |
| 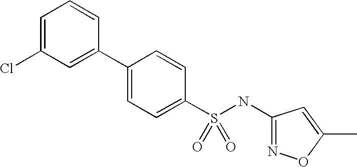 | 0.11 | >20 | 7.7 | 5 | |
| 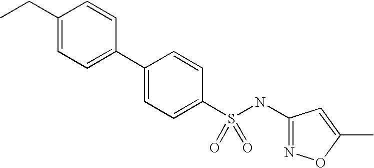 | 0.115 | >20 | −14.4 | 5 | |
| 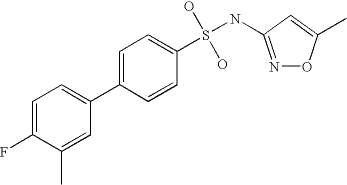 | 0.116 | 1.664, >20 | 2.6 | 5, 1*, 2* | 601.4 |
| 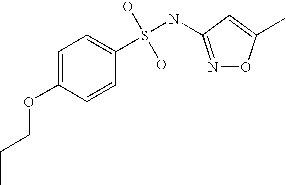 | 0.124 | 8.05 | −11.9 | 5 | |
| 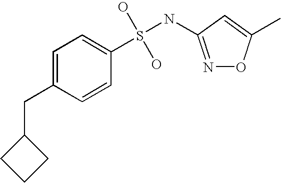 | 0.14 | 8.67 | 4.0 | 5 | |
| 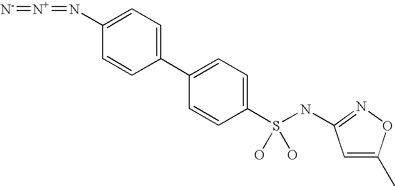 | 0.155 | >20 | 1.1 | 5 | |
| 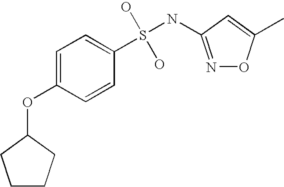 | 0.164 | 9.89 | 9.6 | 4, 5, 2* | |
| 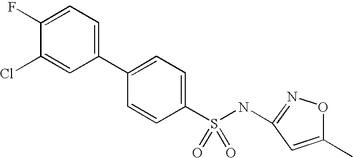 | 0.192 | >20 | 18.6 | 5 | |
| 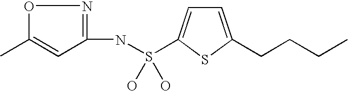 | 0.199 | 8.341, >20 | −26.0 | 5 | |
| 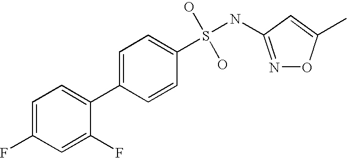 | 0.212, 0.504, 0.659 | 7.35, 18.7, >20 | −3.6 | 5, 1* | >1000 |
| 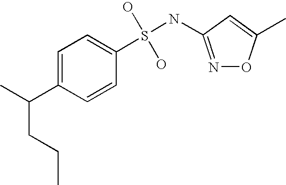 | 0.22 | 18.9 | 5.5 | 5 | |
| 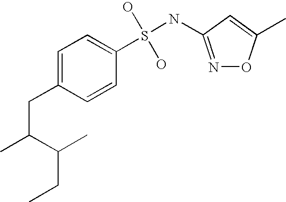 | 0.25 | 10.86 | 15.3 | 4, 5 | |
| 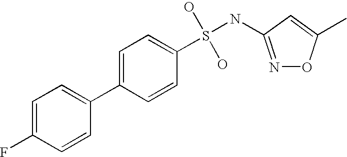 | 0.294 | 3.754, >20 | 12.7 | 1*, 2* | 983.2 |
| 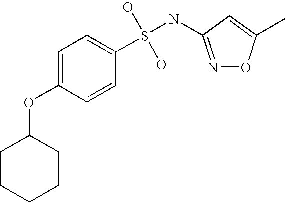 | 0.298 | >20 | 18.2 | 5 | |
| 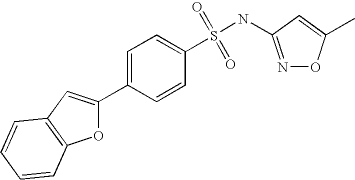 | 0.32 | 2.61 | 23.8 | 2* | |
| 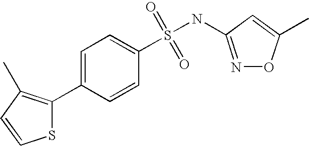 | 0.32 | >20 | 11.5 | 5 | |
| 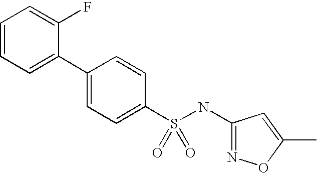 | 0.35 | >20 | −4.3 | 5 | |
| 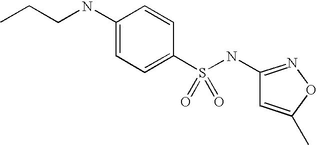 | 0.36033 | 10.367, >20 | −17.9 | 5, 2* | >1000 |
| 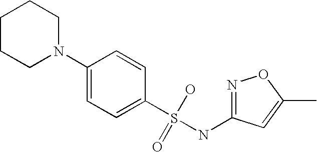 | 0.394 | 4.59 | | 4 | |
| 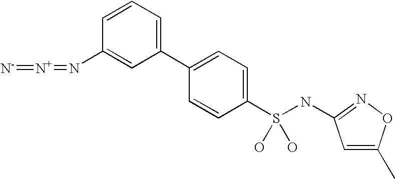 | 0.46 | >20 | 6.0 | 5 | |
| 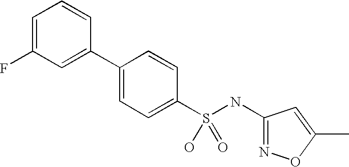 | 0.46 | >20 | −6.4 | 5 | |
| 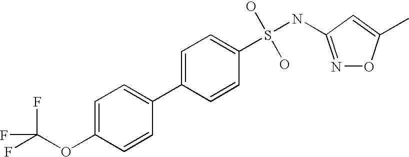 | 0.59 | 5.13 | 19.8 | 5 | |
| 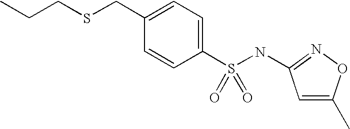 | 0.656 | >20 | −21.3 | 5 | |
| 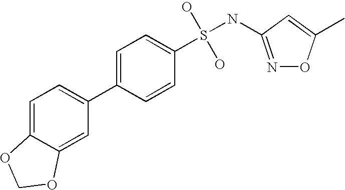 | 0.6736 | >20 | | | >1000 |
| 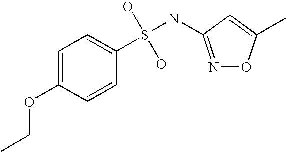 | 0.77 | >20 | −11.5 | 5 | |
| 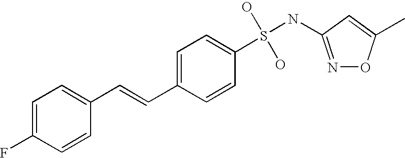 | 0.84 | 11.16 | 4.6 | 5 | |
| 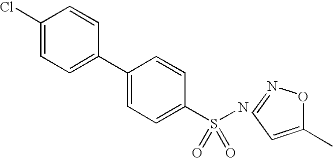 | 0.95 | >20 | 15.9 | 5 | |
| 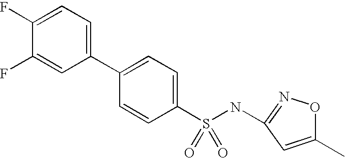 | 1.1 | >20 | 8.8 | 5 | |
| 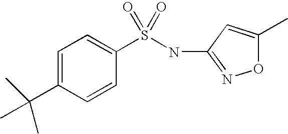 | 1.203 | >20 | −107.0 | 5 | >1000 |
| 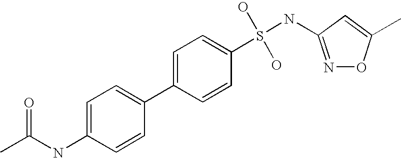 | 1.38 | 19.6 | 3.9 | 5 | |
| 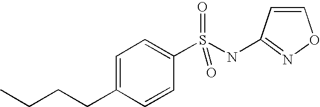 | 1.42 | 17.45 | 18.3 | 5 | |
| 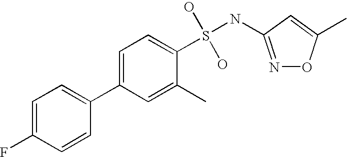 | 1.469 | >20 | 1.3 | 4 | 648.5 |
| 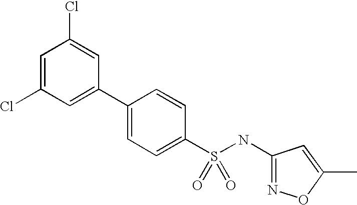 | 1.48 | >20 | −3.1 | 5 | |
| 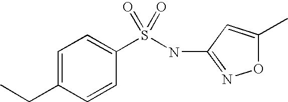 | 1.612 | >20 | −78.9 | 4 | >1000 |
| 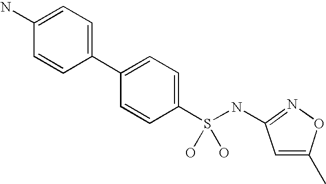 | 1.75 | >20 | −10.2 | 5 | |
| 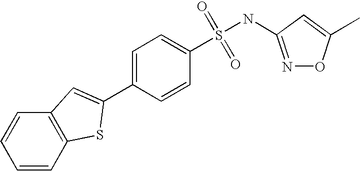 | 1.761 | >20 | −1.8 | 4, 5 | |
| 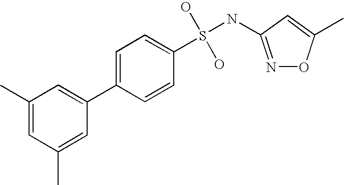 | 1.827 | >20 | −7.3 | 4 | |
| 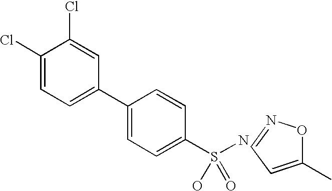 | 1.88 | >20 | 8.7 | 5 | |
| 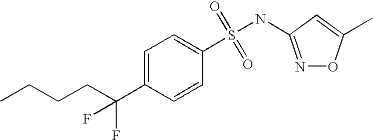 | 2.02 | >20 | −1.6 | 5 | |
| 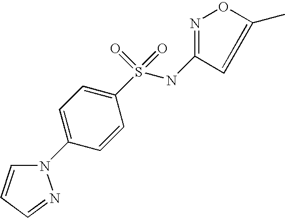 | 2.1 | >20 | −13.3 | 5 | |
| 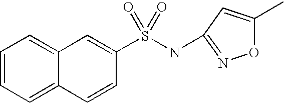 | 2.263 | >20 | −14.7 | 5 | |
| 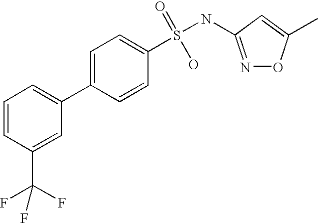 | 2.49 | >20 | 6.9 | 5 | 516.8 |
| 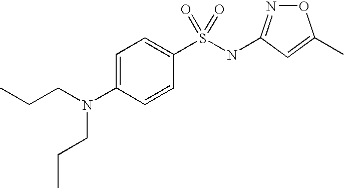 | 2.79 | >20 | −2.8 | 5 | |
| 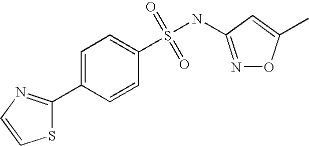 | 2.8 | >20 | 32.7 | 3, 5 | |
| 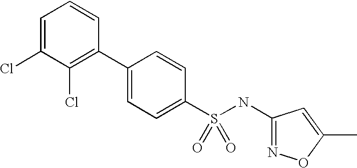 | 3.01 | >20 | −0.3 | 5 | |
| 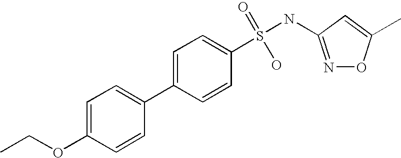 | 3.062 | >20 | −10.5 | 5 | |
| 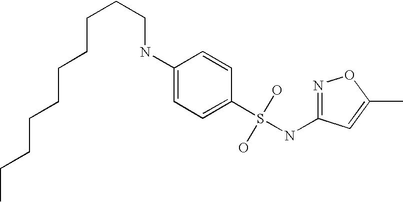 | 3.208 | >20 | 102.8 | 3 | |
| 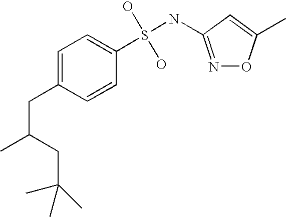 | 3.25 | >20 | −13.8 | 5 | |
| 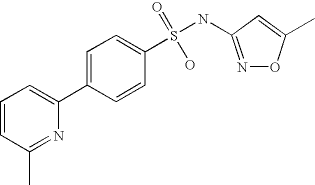 | 3.28 | >20 | −16.1 | 5 | |
| 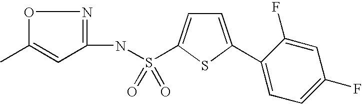 | 3.36 | >20 | −0.6 | 5 | |
| 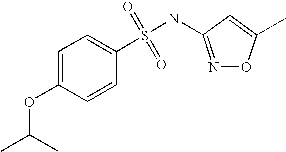 | 3.595 | >20 | 0.8 | 5 | |
| 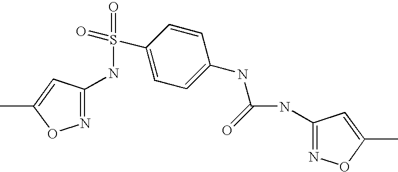 | 5.16 | >20 | −25.4 | 5 | |
| 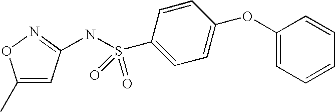 | 5.2055 | >20 | 4.4 | 5 | |
| 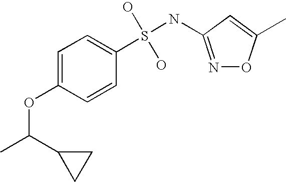 | 5.998 | >20 | −0.9 | 5 | |
| 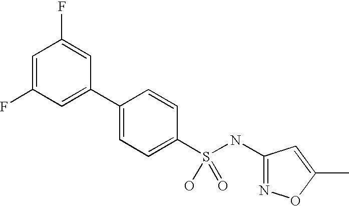 | 6.554 | >20 | 18.6 | 5 | |
| 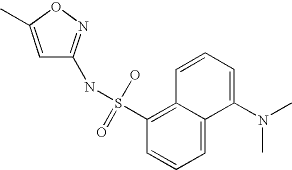 | 6.95 | >20 | 12.4 | 5 | |
| 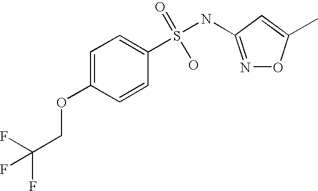 | 7.07 | >20 | 1.8 | 5 | |
| 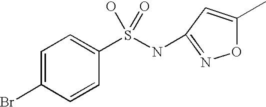 | 7.744 | >20 | −0.7 | 5 | |
| 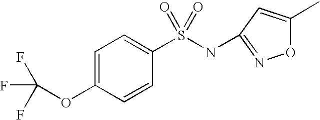 | 10.714 | >20 | 4.0 | 4 | >1000 |
| 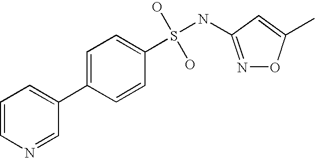 | 11.272 | >20 | 4.5 | 5 | |
| 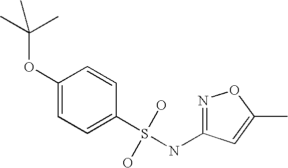 | 12.635 | >20 | −9.7 | 5 | |
| 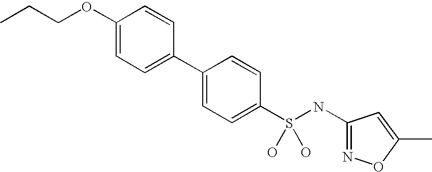 | 14.15 | >20 | 1.4 | 5 | |
| 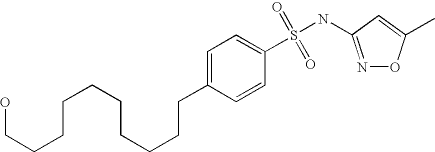 | 17.278 | >20 | 71.0 | 5 | |
| 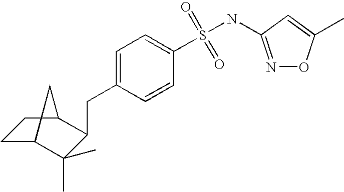 | 17.99 | >20 | −0.6 | 5 | |

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Other embodiments are in the claims.

What is claimed is:

1. A compound having a formula (I): 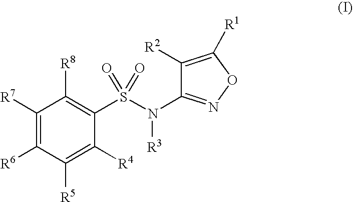
or a pharmaceutically acceptable salt thereof, wherein,
each of R¹ and R² is, independently, H, substituted or unsubstituted C₁₋₆ alkyl, or substituted or unsubstituted C₁₋₆ alkoxy, wherein the substituents are selected from the group consisting of hydroxy and halo;
R³ is H, formyl, acetyl, or substituted or unsubstituted C₁₋₃ alkyl, wherein the substituents are selected from the group consisting of hydroxy and halo;
each of R⁴—R⁸ is, independently, H, halo, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyl)oxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, —NH—C(O)—NH-(substituted or unsubstituted heteroaryl), or —NR¹⁹R²⁰, wherein each of R¹⁹ and R²⁰ is, independently, H, C₁₋₁₂ alkyl, or C₂₋₁₂ alkenyl, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxyalkyloxy, acylamino, acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy;
provided that at least one of R⁴-R⁸ is not H; further provided that when R¹ is alkyl, then R⁶ is not butyl; further provided that when R¹ is methyl and R² is H, then R6 is not —C≡CH, —NHCH₃, CF₃, or —CH₂CH₃.

2. The compound of claim 1, wherein R¹ is C₁-C₄ alkyl.

3. The compound of claim 1, wherein R¹ is CH₃.

4. The compound of claim 1, wherein R⁴, R⁵, R⁷, and R⁸ are H.

5. The compound of claim 1, wherein R³ is H.

6. The compound of claim 1, wherein R⁶ is C₁-C₁₀ alkyl.

7. The compound of claim 6, wherein R⁶ is cyclopentyl.

8. The compound of claim 6, wherein R⁶ is norbornyl.

9. The compound of claim 1, wherein R⁶ is C₁-C₁₀ alkoxy.

10. The compound of claim 1, wherein R⁶ is substituted or unsubstituted C₆-C₁₀ aryl.

11. The compound of claim 1, wherein R⁶ is substituted or unsubstituted C₂-C₁₂ alkenyl.

12. The compound of claim 1, wherein each of R⁴—R⁸ is, independently, H, halo, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, substituted or unsubstituted phenyl, substituted or unsubstituted heteroaryl, or —NH—(C₁₋₆ alkyl), wherein the substituents are selected from the group consisting of hydroxy, halo, and C₁₋₄ alkyl.

13. The compound of claim 12, wherein R⁴, R⁵, R⁷, and R⁸ are H.

14. The compound of claim 12, provided that when R¹ is alkyl, then R⁶ is not alkyl.

15. The compound of claim 12, provided that when R¹ is methyl and R² is H, then R⁶ is not alkyl.

16. The compound of claim 12, provided that when R¹ is methyl and R² is H, then R⁶ is not alkynyl.

17. The compound of claim 12, provided that when R¹ is methyl and R² is H, then R⁶ is not —NH(C₁-C₆ alkyl).

18. The compound of claim 12, wherein R³ is H.

19. A pharmaceutical composition comprising a compound of Formula (I) of claim 1 and a pharmaceutically acceptable carrier.

20. The composition of claim 19, wherein the compound is a compound of claim 2.

21. The composition of claim 19, wherein the compound is a compound of claim 4.

22. The composition of claim 19, wherein the compound is a compound of claim 5.

23. The composition of claim 19, wherein the compound is a compound of claim 6.

24. The composition of claim 19, wherein the compound is a compound of claim 9.

25. The composition of claim 19, wherein the compound is a compound of claim 10.

26. The composition of claim 19, wherein the compound is a compound of claim 12.

27. A method of treating a fungal infection in a subject identified as in need of such treatment comprising administering an effective amount of a compound of Formula (I) in claim 1 to the subject.

28. A method of treating a fungal infection in a subject identified as in need of such treatment comprising administering an effective amount of a pharmaceutical composition of claim 19 to the subject.

29. A compound having a formula (II): 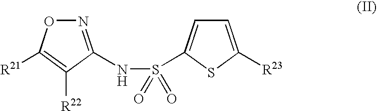
or a pharmaceutically acceptable salt thereof, wherein
each of R²¹ and R²² is, independently, substituted or unsubstituted C₁₋₆ alkyl, or substituted or unsubstituted C₁₋₆ alkoxy, wherein the substituents are selected from the group consisting of hydroxy and halo;
R²³ is substituted or unsubstituted C₁₋₆ alkyl, substituted or unsubstituted C₆₋₁₂ aryl, substituted or unsubstituted C₃₋₁₂ heteroaryl, wherein the substituents are selected from the group consisting of halo, C₁₋₆ alkyl, and C₁₋₆ trihaloalkyl.

30. The compound of claim 29, wherein R²¹ is C₁-C₄ alkyl.

31. The compound of claim 29, wherein R²¹ is CH₃.

32. A pharmaceutical composition comprising a compound of Formula (II) of claim 29 and a pharmaceutically acceptable carrier.

33. A method of treating a fungal infection in a subject identified as in need of such treatment comprising administering an effective amount of a compound of Formula (II) in claim 29 to the subject.

34. A method of treating a fungal infection in a subject identified as in need of such treatment comprising administering an effective amount of a pharmaceutical composition of claim 32 to the subject.

35. A method of treating a fungal infection in a subject identified as in need of such treatment comprising administering a compound of Formula (I) in claim 1 to the subject in combination with a second antimicrobial agent.

36. A composition comprising a compound of Formula (I) in claim 1 and a second antimicrobial agent.

37. A method of treating a fungal infection in a subject identified as in need of such treatment comprising administering a compound of Formula (II) in claim 29 to the subject in combination with a second antimicrobial agent.

38. A composition comprising a compound of Formula (II) in claim 29 and a second antimicrobial agent.

39. A composition according to claim 36 or 38, wherein the second antimicrobial agent is selected from the group consisting of polyenes, candins, sordarins, azoles, allylamines, and morpholines.

40. The method according to claim 35 or 37, wherein the second antimicrobial agent is selected from the group consisting of polyenes, candins, sordarins, azoles, allylamines, and morpholines.
